(19) United States
(12) Patent Application Publication
(10) Pub. No.: US 2004/0014105 A1
(43) Pub. Date: Jan. 22, 2004

(54) Methods for the enrichment of low-abundance polynucleotides

(76) Inventors: Benjamin G. Schroeder, San Mateo, CA (US); Caifu Chen, Palo Alto, CA (US); Gary P. Schroth, San Ramon, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/435,489

(22) Filed: May 9, 2003

Related U.S. Application Data
(63) Continuation-in-part of application No. 10/144,179, filed on May 9, 2002.

(51) Int. Cl. C12Q 1/68; C12P 19/34

(52) U.S. Cl. 435/6; 435/91.2

(57) ABSTRACT

The invention relates to methods for the selective enrichment of low-abundance polynucleotides in a sample. These methods use enzymatically non-extendable nucleobase oligomers to selectively block polymerase activity on high abundance species, thereby resulting in an enrichment of less abundant species in the sample. The invention also relates to the pools of enriched polynucleotides produced by the methods. The resulting pools of enriched polynucleotides find a variety of uses, including the analysis of gene expression and the creation of cDNA libraries.

### REFERENCE TO RELATED APPLICATIONS

The present application claims priority as a continuation-in-part of U.S. patent application Ser. No. 10/144,179, filed May 9, 2002.

### FIELD OF THE INVENTION

The invention relates to compositions and methods for the selective enrichment of low-abundance polynucleotides in a sample. These methods use enzymatically non-extendable nucleobase oligomers to selectively block polymerase activity on high abundance species, thereby resulting in an enrichment of less abundant species in the sample. The resulting pools of enriched polynucleotides find a variety of uses, including the analysis of gene expression and the creation of cDNA libraries.

### INTRODUCTION

The global analysis of gene expression is a formidable challenge for several reasons. One obstacle to the analysis of gene expression is the wide range of expression levels among different genes within a single cell or tissue. It is known that in a single cell type or tissue, two genes can differ in expression levels by more than four orders of magnitude. In contrast, most microarray-based gene expression assays have at most a dynamic range of only two or three orders of magnitude.

Disproportionately few genes account for the majority of expressed cellular mRNA in the pool of mRNA that exists in a cell. These transcripts from highly expressed genes (i.e., genes with a high copy number) are typically “housekeeping” genes that are present in all cell types. The majority of other genes, including metabolic pathway genes, are typically expressed at moderate to low levels (i.e., have lower copy numbers).

Still other genes, in contrast, tend to be expressed at very low levels (i.e., have very low copy numbers). This category of genes includes, for example, genes that encode signal transduction components, including kinases, transcription factors, and cell cycle regulatory proteins. These very low copy number transcripts are often difficult to detect and/or isolate. Ironically, it is these very low copy number transcripts that are most frequently of interest in the study of cell physiology and the molecular basis of human disease. Some of these low-copy number genes show promise in the development of therapeutics for the treatment of disease. Consequently, there is a need to develop compositions and methods for the identification, analysis and/or isolation of low-copy number genes (i.e., low copy number gene transcripts or cDNA molecules).

### SUMMARY OF THE INVENTION

The present invention relates to compositions and methods for the selective enrichment of low-abundance polynucleotides in a sample. These methods use enzymatically non-extendable nucleobase oligomers to selectively block polymerase activity on high abundance species, thereby resulting in an enrichment of less abundant species in the sample. These methods for enrichment of low-abundance species do not require an amplification step; however, in some embodiments, an amplification step can be optionally used. The resulting pools of enriched polynucleotides find a variety of uses, including the analysis of gene expression and the creation of cDNA libraries.

In its broadest aspect, the invention provides methods for the enrichment of a low abundance polynucleotide in a sample of polynucleotides comprising at least one low abundance and at least one high abundance polynucleotide, where the method generally comprises exposing the sample to at least one enzymatically non-extendable nucleobase oligomer having a nucleobase sequence complementary to a sequence within the high abundance polynucleotide under conditions such that base pairing occurs, and then subjecting the sample to conditions for polymerase extension.

A wide variety of enzymatically non-extendable nucleobase oligomers find use with the methods of the invention, and it is not intended that the invention be limited to the type of oligomer used. In one aspect, the enzymatically non-extendable nucleobase oligomer does not have a ribose-containing oligomeric structure. An example of such a structure is a peptide nucleic acid (PNA) oligomer.

In other embodiments, the enzymatically non-extendable nucleobase oligomer is a modified nucleotide oligomer or internucleotide analog oligomer. Examples of such structures include 2′-modified and 3′-modified nucleotide oligomers. More specifically, these structures can include 2′-O-alkyl modified nucleotide oligomers and 3′-alkyl modified nucleotide oligomers. Still more specifically, the 2′-O-alkyl modified nucleotide oligomers can be 2′-O-methyl nucleotide oligomers.

In other embodiments, the modified nucleotide oligomers or internucleotide analog oligomers can be locked nucleic acids (LNA), N3′-P5′ phosphoramidate (NP) oligomers, minor groove binder-linked-oligonucleotides (MGB-linked oligonucleotides), phosphorothioate (PS) oligomers, C₁-C₄ alkylphosphonate oligomers, phosphoramidates, β-phosphodiester oligonucleotides, and a-phosphodiester oligonucleotides. More specifically, the C₁-C₄ alkylphosphonate oligomers can be methyl phosphonate (MP) oligomers.

In still other embodiments, the enzymatically non-extendable nucleobase oligomer used in the methods of the invention is chimeric.

In some embodiments, the invention provides methods for the enrichment of a low abundance polynucleotide in a sample of polynucleotides comprising at least one low abundance and more than one high abundance polynucleotide.

The invention provides methods for the enrichment of a low abundance polynucleotide in a sample of polynucleotides comprising at least one low abundance and at least one high abundance polynucleotide, where the polynucleotides are either RNA or DNA. In some embodiments where the polynucleotides are RNA, the polymerase extension is by reverse transcription and yield a first strand cDNA. In other embodiments, these methods further entail second strand cDNA synthesis. In some embodiments, the sample is exposed to at least one enzymatically non-extendable nucleobase oligomer during first strand cDNA synthesis. Alternatively, the sample is exposed to at least one enzymatically non-extendable nucleobase oligomer during second strand cDNA synthesis. In still other embodiments of these methods, the sample is exposed to at least one enzymatically non-extendable nucleobase oligomer during both first strand cDNA synthesis and second strand cDNA synthesis.

In other embodiments, the methods of the invention for producing a d cDNA can further optionally comprise an amplification step. In some he amplification step is by polymerase chain reaction. In other embodiments, n step is by in vitro transcription.

In some embodiments, the invention provides methods for the enrichment ance polynucleotide in a sample of polynucleotides comprising at least one and at least one high abundance polynucleotide, where the polynucleotide is NA can be mRNA, cRNA or total cellular RNA.

In some embodiments, the invention provides methods for the enrichment ance polynucleotide in a sample of polynucleotides comprising at least one and at least one high abundance polynucleotide, the polynucleotides and polymerase extension is by DNA-dependent DNA-polymerase in a in reaction.

In other embodiments, the methods of the invention for the enrichment of e polynucleotide in a sample of polynucleotides comprising at least one low at least one high abundance polynucleotide further comprise a step of labeling polynucleotides. In some embodiments, the labeling is concomitant with n some embodiments, the labeling is subsequent to amplification.

In other aspects, the invention provides pools of polynucleotides that have for low-abundance polynucleotides. In one embodiment, the invention lity of polynucleotides, where the relative abundance of at least one target has been reduced relative to a non-target polynucleotide, and where at least ucleotide is selected from the list of genes recited in FIG. 14. In a related he invention provides a plurality of polynucleotides, where the relative t least one non-target polynucleotide has been increased relative to a target In one embodiment, the plurality of polynucleotides are either DNA NA molecules. More specifically, the DNA molecules can be cDNA the RNA molecules can be cRNA molecules. In other embodiments, the lynucleotides is labeled. In still other embodiments, the plurality of provided by the invention are cloned into a vector.

In other embodiments, the invention provides kits which facilitate use of the methods provided by the invention. In one embodiment, the invention provides kits for the enrichment of at least one low abundance polynucleotide in a sample of polynucleotides, where the sample comprises at least one high abundance polynucleotide and at least one low abundance polynucleotide, where the kit comprises at least one enzymatically non-extendable nucleobase oligomer having a nucleobase sequence complementary to at least one high abundance target polynucleotide. In some embodiments of these kits, the non-extendable oligomers target a gene or genes recited in FIG. 14.

In other embodiments, the non-extendable nucleobase oligomer provided in the kits is selected from peptide nucleic acid (PNA) oligomers, 2′-O-alkyl modified nucleotide oligomers, 3′-alkyl modified nucleotide oligomers, locked nucleic acids (LNA), N3′-P5′ phosphoramidate (NP) oligomers, minor groove binder-linked-oligonucleotides (MGB-linked oligonucleotides), phosphorothioate (PS) oligomers, C₁-C₄ alkylphosphonate oligomers, phosphoramidates, β-phosphodiester oligonucleotides, and a-phosphodiester oligonucleotides.

In still other embodiments, the kits can optionally comprise various components, such as an RNA-dependent DNA polymerase (reverse transcriptase), a DNA-dependent RNA polymerase, a DNA-dependent DNA polymerase, an oligo-dT polymerase primer, an oligo-dT polymerase primer further comprising nucleotide sequence for RNA polymerase initiation, deoxyribonucleotide triphosphates, ribonucleotide triphosphates, a DNA polymerase primer suitable for cDNA second strand synthesis, and a means for polynucleotide labeling.

In other embodiments, the invention provides methods for analyzing gene expression in a sample having at least one high abundance polynucleotide, where the methods generally comprise the steps of (a) exposing the sample to at least one enzymatically non-extendable nucleobase oligomer having a nucleobase sequence complementary to a sequence within the high abundance polynucleotide under conditions such that base pairing occurs, (b) subjecting the sample to conditions for polymerase extension to produce an enriched polynucleotide sample, (c) labeling the polynucleotides in the enriched polynucleotide sample, (d) contacting the labeled polynucleotide sample with a probe using a hybridization means to form a hybridization complex, and (e) detecting the hybridization complex, where the detection of a hybridization complex is indicative of gene expression.

In other embodiments, the invention provides methods for the synthesis of cDNA libraries enriched for at least one low abundance polynucleotide, generally comprising the steps of (a) providing a sample of mRNA, where the mRNA has at least one high abundance transcript and at least one low abundance transcript, (b) exposing the sample to at least one enzymatically non-extendable nucleobase oligomer having a nucleobase sequence complementary to a sequence within the high abundance mRNA under conditions such that base pairing occurs, (c) subjecting the sample to conditions for reverse transcription and first strand cDNA synthesis, (d) subjecting the sample to conditions for second strand cDNA synthesis to form double stranded cDNA molecules, and (e) cloning the double stranded cDNA molecules into a vector to yield an enriched cDNA library.

### BRIEF DESCRIPTION OF THE DRAWINGS

FIG. 1 shows a graph depicting the results of a serial analysis of gene expression (SAGE). The X-axis plots the SAGE Tag ID (10-mer oligonucleotides), and the Y-axis plots the frequency of appearance of a particular Tag.

FIG. 2 shows a hypothetical analysis of gene expression and hybridization, where seven different gene transcripts having a 100,000-fold range in expression are analyzed. The calculations utilize a range of 0.1-500 μg of unamplified cellular mRNA in a 250 μL hybridization reaction. The predicted concentrations of each of the gene transcripts in the hybridization reaction are provided in pM.

FIG. 3 shows a table providing hypothetical calculations of mRNA quantitation and concentration in a 250 μL array hybridization, given different amounts of starting material varying from 10⁴ through 10⁸ HeLa cells. Assuming an average transcript length of 1.9 kilobases (kb), the table provides the hypothetical RNA yield (in μg, pmol and number of molecules) and the predicted mRNA molar concentration in a hybridization reaction. These calculations are shown for low, intermediate and high abundance classes of mRNA transcript. In the table, mRNA species above a 1 pM lower limit of detection are shown in boxes.

FIG. 4 shows a hypothetical analysis of gene expression and hybridization, where six different genes (genes A-F) having a 10,000-fold range in levels of expression are amplified and analyzed in a hybridization method. Three scenarios are provided, where 1, 10 or 100 μg of either labeled cDNA or cRNA are used in the hybridization reactions. The predicted concentrations of each of the gene transcripts in the hybridization reaction are provided in pM.

FIG. 5 shows a hypothetical gene expression analysis similar to FIG. 4, with the exception that the level of the most abundant transcript (gene A) has been reduced by 99%.

FIG. 6 shows the PCR amplicon nucleotide sequence of the human import precursor of subunit B of the H⁺ transporting, mitochondrial ATP synthase, subunit B, isoform 1 (ATP5F1) gene. The region of the PCR amplicon used as a synthetic RNA template is shown underlined.

FIG. 7 shows the PCR amplicon nucleotide sequence of the human cholesteryl ester transfer protein (CETP) gene. The region of the PCR amplicon used as a synthetic RNA template is shown underlined.

FIG. 8 shows a table describing 18 different synthetic PNA oligomers (numbers 858-875) specific and complementary in sequence to the human ATP5F1 gene transcript. The sequence and position of the PNA oligomers is provided. The predicted Tₘ (° C.) of the PNA:RNA duplex is also shown, as well as the predicted Tₘ of an analogous oligodeoxyribonucleotide having the same base sequence as the PNA oligonucleotide. “O” positions in the sequences indicate a linker/spacer, the structure of which is shown in FIG. 10.

FIG. 9 shows a table describing 19 different synthetic PNA oligomers (numbers 839-857) specific and complementary in sequence to the human CETP gene transcript. The sequence and position of the PNA oligomers is provided. The predicted Tₘ (° C.) of the PNA:RNA duplex is also shown, as well as the predicted Tₘ of an analogous oligodeoxyribonucleotide having the same base sequence as the PNA oligonucleotide. “O” positions in the sequences indicate a linker/spacer, the structure of which is shown in FIG. 10.

FIGS. 10A through 10C show the structure of the GEN063032 linker/spacer. FIG. 10A shows the structure of this molecule when it is at an internal position in a PNA oligomer. FIG. 10B shows the structure of the molecule when it is in an amino-terminal position within a PNA oligomer molecule. FIG. 10C shows the structure of the molecule when it is in a carboxy-terminal position within a PNA oligomer molecule.

FIG. 11 shows an image of an ethidium bromide-stained agarose gel, containing the single-stranded products of various reverse transcriptase reactions (i.e., RT first strand synthesis; lanes 2-10). These RT reactions used an ATP5F1 synthetic RNA template, an oligo-dT synthetic primer, and various ATP5F1-specific PNA blocking oligomers. Also on the gel are control reactions containing only template RNA (lane 12), primerless RT reaction (lane 11) and 1-Kb DNA ladder (lane 1).

FIG. 12 shows an image of an ethidium bromide-stained agarose gel, containing the single-stranded products of various reverse transcriptase reactions (i.e., RT first strand synthesis; lanes 2-7). These RT reactions used an ATP5F1 synthetic RNA template, an oligo-dT synthetic primer, and a concentration titration of ATP5F1-specific PNA blocking oligonucleotide number 864. Also on the gel are control reactions containing only template RNA (lane 10), primerless RT reaction (lane 9), NMP-buffer control (lane 8), 1-Kb DNA ladder (lane 1) and an RNA size ladder (lane 11).

FIG. 13 shows an image of an ethidium bromide-stained agarose gel, containing the single-stranded products of various reverse transcriptase reactions (i.e., RT first strand synthesis; lanes 2-7). These RT reactions used an CETP synthetic RNA template, an oligo-dT synthetic primer, and a concentration titration of ATP5F1-specific PNA blocking oligonucleotide number 864. Also on the gel are control reactions containing only template RNA (lane 10), primerless RT reaction (lane 9), NMP-buffer control (lane 8), 1-Kb DNA ladder (lane 1) and an RNA size ladder (lane 11).

FIG. 14 provides a table of known highly expressed genes, along with GenBank Accession numbers for the expressed cDNA sequences of those genes.

FIG. 15 shows the results of a TaqMan® quantitative RT-PCR analysis of six cRNA products generated by in vitro transcription of cDNA molecules derived from either total cellular RNA or mRNA isolated from human liver. The reverse transcriptase reaction that generated the cDNA pool was run either in the absence or presence of blocking PNA oligonspecific for the ATP5F1 and CETP genes. Values shown in the table are threshold cycles (C_{T}). Quantitation of cRNA was determined for both targeted and non-targeted

FIG. 16 shows a graphical representation of the threshold cycle (C_{T}) Taqman® analysis data shown in FIG. 15. The open bars represents C_{T} values generated using cRNA synthesized from cDNA derived mRNA in the absence of any blocking PNA oligomers, the speckled bar represents C_{T} values generated using cRNA synthesized from cDNA derived from mRNA in the presence of blocking PNA oligomers, the striped bar represents C_{T} values generated using cRNA synthesized from cDNA derived from total RNA in the absence of any blocking PNA oligomers, and the solid bar represents C_{T} values generated using cRNA synthesized from cDNA derived from total RNA in the presence of blocking PNA oligomers.

FIG. 17 shows a flow chart of cDNA synthesis and other aspects of the presnet invention. The use of blocking oligomers in these various reactions is indicated by a large arrow.

### DETAILED DESCRIPTION OF THE INVENTION

Definitions

Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Nucleobase” means any nitrogen-containing heterocyclic moiety capable of forming Watson-Crick hydrogen bonds in pairing with a complementary nucleobase or nucleobase analog (i.e., derivatives of nucleobases). “Heterocyclic” refers to a molecule with a ring system in which one or more ring atom is a heteroatom, e.g., nitrogen, oxygen, or sulfur (i.e., not carbon). A large number of nucleobases, nucleobase analogs and nucleobase derivatives are known. Examples of nucleobases include purines and pyrimidines, and modified forms, e.g., 7-deazapurine. Typical nucleobases are the naturally occurring nucleobases adenine, guanine, cytosine, uracil, thymine, and analogs (Seela, U.S. Pat. No. 5,446,139) of the naturally occurring nucleobases, e.g., 7-deazaadenine, 7-deazaguanine, 7-deaza-8-azaguanine, 7-deaza-8-azaadenine, inosine, nebularine, nitropyrrole (Bergstrom, J. Amer. Chem. Soc., 117:1201-1209 [1995]), nitroindole, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine (Seela, U.S. Pat. No. 6,147,199), 7-deazaguanine (Seela, U.S. Pat. No. 5,990,303), 2-azapurine (Seela, WO 01/16149), 2-thiopyrimidine, 6-thioguanine, 4-thiothymine, 4-thiouracil, O⁶-methylguanine, N6-methyladenine, O⁴-methylthymine, 5,6-dihydrothymine, 5,6-dihydrouracil, 4-methylindole, pyrazolo[3,4-D]pyrimidines, “PPG” (Meyer, U.S. Pat. Nos. 6,143,877 and 6,127,121; Gall, WO 01/38584), and ethenoadenine (Fasman (1989) in Practical Handbook of Biochemistry and Molecular Biology, pp. 385-394, CRC Press, Boca Raton, Fla.).

The term “nucleobase oligomer” or “oligomer” as used herein refers to a polymeric arrangement of nucleobases. An oligomer can be single- or double-stranded, and can be complementary to the sense or antisense strand of a gene sequence. A nucleobase oligomer can hybridize with a complementary portion of a target polynucleotide to form a duplex, which can be a homoduplex or a heteroduplex. A nucleobase oligomer is short, typically but not exclusively, less than 100 nucleobases in length. Linkages between nucleobases can be internucleotide-type phosphodiester linkages, or any other type of linkage. A nucleobase oligomer can be enzymatically extendable or enzymatically non-extendable.

“Nucleoside” refers to a compound consisting of a nucleobase linked to the C-1′ carbon of a sugar, such as ribose, arabinose, xylose, and pyranose, in the natural β or the α anomeric configuration. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Ribose examples include ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides (Asseline et al., Nucl. Acids Res., 19:4067-74 [1991]), 2′-4′- and 3′-4′-linked and other “locked” or “LNA”, bicyclic sugar modifications (WO 98/22489; WO 98/39352; WO 99/14226). Exemplary LNA sugar analogs within a polynucleotide include the structures:

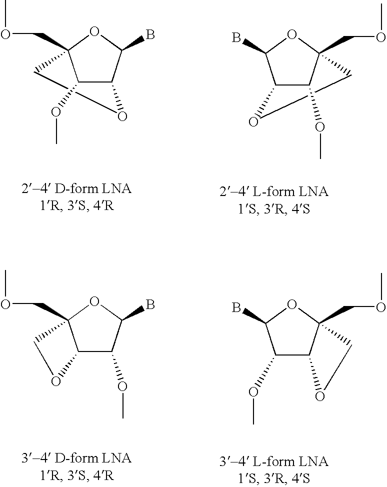

where B is any nucleobase.

Sugars include modifications at the 2′- or 3′-position such as methoxy, ethoxy, allyloxy, isopropoxy, butoxy, isobutoxy, methoxyethyl, alkoxy, phenoxy, azido, amino, alkylamino, fluoro, chloro and bromo. Nucleosides and nucleotides include the natural D configurational isomer (D-form), as well as the L configurational isomer (L-form) (Beigelman, U.S. Pat. No. 6,251,666; Chu, U.S. Pat. No. 5,753,789; Shudo, EP0540742; Garbesi et al., Nucl. Acids Res., 21:4159-4165 (1993); Fujimori, J. Amer. Chem. Soc., 112:7435 (1990); Urata, (1993) Nucleic Acids Symposium Ser. No. 29:69-70). When the nucleobase is purine, e.g., A or G, the ribose sugar is attached to the N⁹-position of the nucleobase. When the nucleobase is pyrimidine, e.g., C, T or U, the pentose sugar is attached to the N′-position of the nucleobase (Kornberg and Baker, (1992) DNA Replication, 2^{nd} Ed., Freeman, San Francisco, Calif.).

“Nucleotide” refers to a phosphate ester of a nucleoside, as a monomer unit or within a polynucleotide. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g., α-thio-nucleotide 5′-triphosphates. For a review of polynucleotide and nucleic acid chemistry, see Shabarova, Z. and Bogdanov, A. Advanced Organic Chemistry of Nucleic Acids, VCH, New York, 1994.

As used herein, the terms “polynucleotide” and “oligonucleotide” are used interchangeably and mean single-stranded and double-stranded polymers of nucleotide monomers, including 2′-deoxyribonucleotides (DNA) and ribonucleotides (RNA) linked by internucleotide phosphodiester bond linkages, e.g., 3′-5′ and 2′-5′, inverted linkages, e.g., 3′-3′ and 5′-5′, branched structures, or internucleotide analogs. A “polynucleotide sequence” refers to the sequence of nucleotide monomers along the polymer.

The term “RNA” is used broadly and includes, for example and without limitation, RNA, cRNA, rRNA, mRNA and tRNA.

Polynucleotides that are formed by 3′-5′ phosphodiester linkages are said to have 5′-ends and 3′-ends because the mononucleotides that are reacted to make the polynucleotide are joined in such a manner that the 5′ phosphate of one mononucleotide pentose ring is attached to the 3′ oxygen (i.e., hydroxyl) of its neighbor in one direction via the phosphodiester linkage. Thus, the 5′-end of a polynucleotide molecule has a free phosphate group or a hydroxyl at the 5′ position of the pentose ring of the nucleotide, while the 3′ end of the polynucleotide molecule has a free phosphate or hydroxyl group at the 3′ position of the pentose ring. Within a polynucleotide molecule, a position or sequence that is oriented 5′ relative to another position or sequence is said to be located “upstream,” while a position that is 3′ to another position is said to be “downstream.” This terminology reflects the fact that polymerases proceed and extend a polynucleotide chain in a 5′ to 3′ fashion along the template strand.

Polynucleotides have associated counter ions, such as H⁺, NH₄⁺, trialkylammonium, Mg²⁺, Na⁺ and the like. A polynucleotide may be composed entirely of deoxyribonucleotides, entirely of ribonucleotides, or chimeric mixtures thereof. Polynucleotides may be comprised of internucleotide, nucleobase and sugar analogs. Unless denoted otherwise, whenever a polynucleotide sequence is represented, it will be understood that the nucleotides are in 5′ to 3′ orientation from left to right and that “A” denotes deoxyadenosine, “C” denotes deoxycytidine, “G” denotes deoxyguanosine, and “T” denotes thymidine, unless otherwise noted.

“Polynucleotides” are not limited to any particular length of nucleotide sequence, as the term “polynucleotides” encompasses polymeric forms of nucleotides of any length. Polynucleotides that range in size from about 5 to about 40 monomeric units are typically referred to in the art as oligonucleotides. Polynucleotides that are several thousands or more monomeric nucleotide units in length are typically referred to as nucleic acids. Polynucleotides can be linear, branched linear, or circular molecules.

As used herein, the terms “complementary” or “complementarity” are used in reference to antiparallel strands of nucleobases (i.e., a sequence of nucleobases) related by the Watson/Crick and Hoogsteen-type base-pairing rules. For example, the sequence 5′-AGTTC-3′ is complementary to the sequence 5′-GAACT-3′.

As used herein, the term “antisense” refers to any polynucleotide or other nucleobase oligomer which is antiparallel to and complementary to another nucleobase oligomer. The term “complementary” is sometimes used interchangeably with “antisense.” The present invention encompasses antisense DNA, RNA or any other nucleobase oligomer produced by any method.

As used herein, the term “Tₘ” is used in reference to the “melting temperature.” The melting temperature is the temperature at which a population of double-stranded polynucloetide molecules or nucleobase oligomers, in homoduplexes or heteroduplexes, become half dissociated into single strands. The equation for calculating the Tₘ between two molecules takes into account the base sequence as well as other factors including structural and sequence characteristics and nature of the oligomeric linkages. Methods for determining Tₘ are known in the art.

“Internucleotide analog” means a phosphate ester analog or a non-phosphate analog of a polynucleotide. Phosphate ester analogs include: (i) C₁-C₄ alkylphosphonate, e.g., methylphosphonate; (ii) phosphoramidate; (iii) C₁-C₆ alkyl-phosphotriester; (iv) phosphorothioate; and (v) phosphorodithioate.

Non-phosphate internucleotide analogs include the family of peptide nucleic acids, commonly referred to as PNA, in which the sugar/phosphate backbone of DNA or RNA has been replaced with acyclic, achiral, and neutral polyamide linkages (U.S. Pat. No. 5,539,082; WO 92/20702; Nielsen et al., Science 254:1497-1500 [1991]; Egholm et al., Nature 365:566-568 [1993]). The 2-aminoethylglycine polyamide linkage with nucleobases attached to the linkage through an amide bond has been well-studied as one embodiment of PNA and shown to possess exceptional hybridization specificity and affinity. A partial structure of this molecule is shown below with a carboxyl-terminal amide, and where B is any nucleobase:

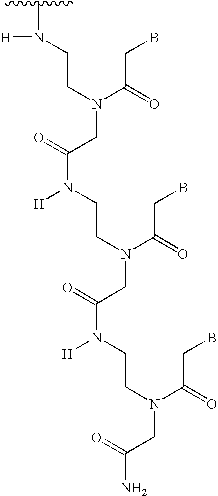

Despite its name, PNA is neither truly a peptide, a nucleic acid, nor acidic. PNA is a non-naturally occurring molecule, and is not known to be a substrate for any polymerase enzyme, peptidase or nuclease. Because a PNA is a polyamide, it has a C-terminus (carboxyl terminus) and an N-terminus (amino terminus). For the purposes of the design of a PNA oligomer suitable for antiparallel binding (i.e., hybridization) to a target sequence, the N-terminus of the nucleobase sequence of the PNA oligomer is the equivalent of the 5′-hydroxyl terminus of an equivalent DNA or RNA oligonucleotide. As used herein, it is intended that the term “PNA” also include related structures as known in the art, especially other peptide-based nucleic acid mimics (see, e.g., WO 96/04000).

Methods for the synthesis of PNAs are known in the art (see, e.g., Hyrup and Nielsen, Bioorg. Med. Chem., 4(1):5-23 (1996); WO 92/20702; WO 92/20703 and U.S. Pat. No. 5,539,082). Chemical assembly of PNA oligomers is analogous to solid phase peptide synthesis, wherein at each cycle of assembly the oligomer possesses a reactive alkyl amino-terminus that is condensed with the next monomer unit to be added to the growing oligomer. Because standard peptide chemistry is utilized, natural and non-natural amino acids can be incorporated into a PNA oligomer, and can be synthesized using tBoc or Fmoc solid phase synthesis. Chemical reagents and instrumentation for support-bound automated chemical synthesis of PNA oligomers are commercially available, and PNA oligomers having custom nucleobase sequences are readily ordered from commercial vendors (e.g., Applied Biosystems, Foster City, Calif.).

“Substituted” as used herein refers to a molecule wherein one or more hydrogen atoms are replaced with one or more non-hydrogen atoms, functional groups or moieties. For example, an unsubstituted nitrogen is —NH₂, while a substituted nitrogen is —NHCH₃. Exemplary substituents include but are not limited to halo, e.g., fluorine and chlorine, C₁-C₈ alkyl, sulfate, sulfonate, sulfone, amino, ammonium, amido, nitrile, nitro, alkoxy (—OR where R is C₁-C₁₂ alkyl), phenoxy, aromatic, phenyl, polycyclic aromatic, heterocycle, water-solubilizing group, and linking moiety.

“Alkyl” means a saturated or unsaturated, straight-chain, branched, cyclic, or substituted hydrocarbon radical derived by the removal of one hydrogen atom from a single carbon atom of a parent alkane, alkene, or alkyne. Typical alkyl groups consist of 1-12 saturated and/or unsaturated carbons, including, but not limited to, methyl, ethyl, cyanoethyl, isopropyl, butyl, and the like.

“Alkyldiyl” means a saturated or unsaturated, branched, straight chain, cyclic, or substituted hydrocarbon radical of 1-12 carbon atoms, and having two monovalent radical centers derived by the removal of two hydrogen atoms from the same or two different carbon atoms of a parent alkane, alkene or alkyne. Typical alkyldiyl radicals include, but are not limited to, 1,2-ethyldiyl (—CH₂CH₂—), 1,3-propyldiyl (—CH₂CH₂CH₂—), 1,4-butyldiyl (—CH₂CH₂CH₂CH₂—), and the like. “Alkoxydiyl” means an alkoxyl group having two monovalent radical centers derived by the removal of a hydrogen atom from the oxygen and a second radical derived by the removal of a hydrogen atom from a carbon atom. Typical alkoxydiyl radicals include, but are not limited to, methoxydiyl (—OCH₂—) and 1,2-ethoxydiyl or ethyleneoxy (—OCH₂CH₂—). “Alkylaminodiyl” means an alkylamino group having two monovalent radical centers derived by the removal of a hydrogen atom from the nitrogen and a second radical derived by the removal of a hydrogen atom from a carbon atom. Typical alkylaminodiyl radicals include, but are not limited to —NHCH₂—, —NHCH₂CH₂—, and —NHCH₂CH₂CH₂—. “Alkylamidediyl” means an alkylamide group having two monovalent radical centers derived by the removal of a hydrogen atom from the nitrogen and a second radical derived by the removal of a hydrogen atom from a carbon atom. Typical alkylamidediyl radicals include, but are not limited to —NHC(O)CH₂—, —NHC(O)CH₂CH₂—, and —NHC(O)CH₂CH₂CH₂—.

“Aryl” means a monovalent aromatic hydrocarbon radical of 5-14 carbon atoms derived by the removal of one hydrogen atom from a single carbon atom of a parent aromatic ring system. Typical aryl groups include, but are not limited to, radicals derived from benzene, substituted benzene, naphthalene, anthracene, biphenyl, and the like, including substituted aryl groups.

“Aryldiyl” means an unsaturated cyclic or polycyclic hydrocarbon radical of 5-14 carbon atoms having a conjugated resonance electron system and at least two monovalent radical centers derived by the removal of two hydrogen atoms from two different carbon atoms of a parent aryl compound, including substituted aryldiyl groups.

“Substituted alkyl”, “substituted alkyldiyl”, “substituted aryl” and “substituted aryldiyl” mean alkyl, alkyldiyl, aryl and aryldiyl respectively, in which one or more hydrogen atoms are each independently replaced with another substituent. Typical substituents include, but are not limited to, F, Cl, Br, I, R, OH, —OR, —SR, SH, NH₂, NHR, NR₂, —+NR₃, —N═NR₂, —CX₃, —CN, —OCN, —SCN, —NCO, —NCS, —NO, —NO₂, —N₂⁺, —N₃, —NHC(O)R, —C(O)R, —C(O)NR₂—S(O)₂O⁻, —S(O)₂R, —OS(O)₂OR, —S(O)₂NR, —S(O)R, —OP(O)(OR)₂, —P(O)(OR)₂, —P(O)(O⁻)₂, —P(O)(OH)₂, —C(O)R, —C(O)X, —C(S)R, —C(O)OR, —CO₂⁻, —C(S)OR, —C(O)SR, —C(S)SR, —C(O)NR₂, —C(S)NR₂, —C(NR)NR₂, where each R is independently —H, C₁-C₆ alkyl, C₅-C₁₄ aryl, heterocycle, or linking group. Substituents also include divalent, bridging functionality, such as diazo (—N═N—), ester, ether, ketone, phosphate, alkyldiyl, and aryldiyl groups.

As used herein, “enzymatically extendable” as it applies to a nucleobase oligomer refers to a nucleobase oligomer that capable of serving as an enzymatic substrate for the incorporation (i.e., extension) of nucleotides complementary to a polynucleotide template by a polymerase enzyme. An enzymatically extendable nucleobase oligomer can serve as a polymerase “primer” and supports primer extension. Examples of enzymatically extendable nucleobase oligomers includes oligomers comprising 2-deoxyribose polynucleotides (DNA) and ribose polynucleotides (RNA), where the oligomers have a free ribose sugar 3′ hydroxyl group.

As used herein, “enzymatically non-extendable” as it applies to a nucleobase oligomer refers to a nucleobase oligomer that is incapable of serving as an enzymatic substrate for the incorporation (i.e., extension) of nucleotides complementary to a polynucleotide template by a polymerase enzyme. An enzymatically non-extendable nucleobase oligomer can not serve as a polymerase “primer” and can not initiate primer extension. Numerous examples of enzymatically non-extendable nucleobase oligomer structures are known in the art. These structures include, for example, any polynucleotide that: (i) is lacking a hydroxyl group on the 3′ position of the ribose sugar in the 3′ terminal nucleotide, (ii) has a modification to a sugar, nucleobase, or internucleotide linkage at or near the 3′ terminal nucleotide that blocks polymerase activity, e.g., 2′-O-methyl; or (iii) nucleobase oligomers that do not utilize a ribose sugar phosphodiester backbone in their oligmeric structure. Examples of the latter include, but are not limited to, peptide nucleic acids, termed PNAs. As used herein, the terms “non-extendable oligomer” and “blocking oligomer” are used interchangeably.

Non-extendable nucleobase oligomers can be formed by using “terminator nucleotides.” Terminator nucleotides are nucleotides that are capable of being enzymatically incorporated onto a 3′ terminus of a polynucleotide through the action of a polymerase enzyme, but cannot be further extended. Thus, a terminator nucleotide is enzymatically incorporatable, but not enzymatically extendable. Examples of terminator nucleotides include 2,3-dideoxyribonucleotides (ddNTP), 2′-deoxy, 3′-fluoro nucleotide 5′-triphosphates, and labelled forms thereof.

As used herein, “target”, “target polynucleotide”, and “target sequence” and the like refer to a specific polynucleotide sequence that is the subject of hybridization with a complementary polynucleotide, e.g., a blocking oligomer, or a cDNA first strand synthesis primer. The target sequence can be composed of DNA, RNA, analogs thereof, or combinations thereof. The target can be single-stranded or double-stranded. In primer extension processes, the target polynucleotide which forms a hybridization duplex with the primer may also be referred to as a “template.” A template serves as a pattern for the synthesis of a complementary polynucleotide (Concise Dictionary of Biomedicine and Molecular Biology, (1996) CPL Scientific Publishing Services, CRC Press, Newbury, UK). A target sequence for use with the present invention may be derived from any living or once living organism, including but not limited to prokaryote, eukaryote, plant, animal, and virus, as well as synthetic and/or recombinant target sequences.

As used herein, the term “probe” refers to a polynucleotide that is capable of forming a duplex structure by complementary base pairing with a sequence of a target polynucleotide. Subsequently, the duplex so formed is detected, visualized, measured and/or quantitated. In some embodiments, the probe is fixed to a solid support, such as in a chip array format.

As used herein, the term “primer” refers to an oligonucleotide of defined sequence that is designed to hybridize with a complementary, primer-specific portion of a target sequence and undergo primer extension. A primer can function as the starting point for the enzymatic polymerization of nucleotides, which may be referred to as primer extension (Concise Dictionary of Biomedicine and Molecular Biology, (1996) CPL Scientific Publishing Services, CRC Press, Newbury, UK).

The term “duplex” means an intermolecular or intramolecular double-stranded portion of one or more nucleobase oligomers which is base-paired through Watson-Crick, Hoogsteen, or other sequence-specific interactions of nucleobases. In one embodiment, a duplex may consist of a primer and a template strand. In another embodiment, a duplex may consist of a non-extendable nucleobase oligomer and a target strand. A “hybrid” means a duplex, triplex, or other base-paired complex of nucleobase oligomers interacting by base-specific interactions, i.e., Watson-Crick or Hoogsteen type interactions.

The term “primer extension” means the process of elongating an extendable primer that is annealed to a target in the 5′ to 3′ direction using a template-dependent polymerase. The extension reaction uses appropriate buffers, salts, pH, temperature, and nucleotide triphosphates, including analogs and derivatives thereof, and a template-dependent polymerase. Suitable conditions for primer extension reactions are well known in the art. The template-dependent polymerase incorporates nucleotides complementary to the template strand starting at the 3′-end of an annealed primer, to generate a complementary strand.

As used herein, the term “label” in reference to polynucleotides refers to any moiety which can be attached to a polynucleotide and: (i) provides a detectable signal; (ii) interacts with a second label to modify the detectable signal provided by the second label, e.g., FRET; (iii) stabilizes hybridization, i.e., duplex formation; (iv) confers a capture function, i.e., hydrophobic affinity, antibody/antigen, ionic complexation, or (v) changes a physical property, such as electrophoretic mobility, hydrophobicity, hydrophilicity, solubility, or chromatographic behavior. Labeling can be accomplished using any one of a large number of known techniques employing known labels, linkages, linking groups, reagents, reaction conditions, and analysis and purification methods. Labels include light-emitting or light-absorbing compounds which generate or quench a detectable fluorescent, chemiluminescent, or bioluminescent signal (Kricka, L. in Nonisotopic DNA Probe Techniques (1992), Academic Press, San Diego, pp. 3-28). Fluorescent reporter dyes useful for labelling biomolecules include fluoresceins (U.S. Pat. Nos. 5,188,934; 6,008,379; 6,020,481), rhodamines (U.S. Pat. Nos. 5,366,860; 5,847,162; 5,936,087; 6,051,719; 6,191,278), benzophenoxazines (U.S. Pat. No. 6,140,500), energy-transfer dye pairs of donors and acceptors (U.S. Pat. Nos. 5,863,727; 5,800,996; 5,945,526), and cyanines (Kubista, WO 97/45539), as well as any other fluorescent label capable of generating a detectable signal. Examples of fluorescein dyes include 6-carboxyfluorescein; 2′,4′,1,4,-tetrachlorofluorescein; and 2′,4′,5′,7′, 1,4-hexachlorofluorescein (Menchen, U.S. Pat. No. 5,118,934).

Another class of labels are hybridization-stabilizing moieties which serve to enhance, stabilize, or influence hybridization of duplexes, e.g., intercalators, minor-groove binders, and cross-linking functional groups (Blackburn, G. and Gait, M. Eds. “DNA and RNA structure” in Nucleic Acids in Chemistry and Biology, 2^{nd} Edition, (1996) Oxford University Press, pp. 15-81). Yet another class of labels effect the separation or immobilization of a molecule by specific or non-specific capture, for example biotin, digoxigenin, and other haptens (Andrus, A. “Chemical methods for 5′ non-isotopic labelling of PCR probes and primers” (1995) in PCR 2: A Practical Approach, Oxford University Press, Oxford, pp. 39-54). Non-radioactive labelling methods, techniques, and reagents are reviewed in: Non-Radioactive Labelling, A Practical Introduction, Garman, A. J. (1997) Academic Press, San Diego.

The terms “annealing” and “hybridization” are used interchangeably and mean the base-pairing interaction of one polynucleotide with another polynucleotide that results in formation of a duplex or other higher-ordered structure. The primary interaction is base specific, i.e., A/T and G/C, by Watson/Crick and Hoogsteen-type hydrogen bonding.

The term “solid support” refers to any solid phase material upon which an oligonucleotide is synthesized, attached or immobilized. Solid support encompasses terms such as “resin”, “solid phase”, and “support”. A solid support may be composed of organic polymers such as polystyrene, polyethylene, polypropylene, polyfluoroethylene, polyethyleneoxy, and polyacrylamide, as well as co-polymers and grafts thereof. A solid support may also be inorganic, such as glass, silica, controlled-pore-glass (CPG), or reverse-phase silica. The configuration of a solid support may be in the form of beads, spheres, particles, granules, a gel, or a surface. Surfaces may be planar, substantially planar, or non-planar. Solid supports may be porous or non-porous, and may have swelling or non-swelling characteristics. A solid support may be configured in the form of a well, depression or other container, vessel, feature or location. A plurality of solid supports may be configured in an array at various locations, addressable for robotic delivery of reagents, or by detection means including scanning by laser illumination and confocal or deflective light gathering.

As used herein, “array” or “microarray” mean a predetermined spatial arrangement of hybridizable elements (e.g., polynucleotides) present on a solid support and/or in an arrangement of vessels. Certain array formats are referred to as a “chip” or “biochip” (M. Schena, Ed. Microarray Biochip Technology, BioTechnique Books, Eaton Publishing, Natick, MA [2000]). An array can comprise a low-density number of addressable locations, e.g., 2 to about 12, medium-density, e.g., about a hundred or more locations, or a high-density number, e.g., a thousand or more. Typically, the array format is a geometrically-regular shape which allows for facilitated fabrication, handling, placement, stacking, reagent introduction, detection, and storage. The array may be configured in a row and column format, with regular spacing between each location. Alternatively, the locations may be bundled, mixed, or homogeneously blended for equalized treatment or sampling. An array may comprise a plurality of addressable locations configured so that each location is spatially addressable for high-throughput handling, robotic delivery, masking, or sampling of reagents. An array can also be configured to facilitate detection or quantitation by any particular means, including but not limited to, scanning by laser illumination, confocal or deflective light gathering, and chemical luminescence. In its broadest sense, “array” formats, as recited herein, include but are not limited to, arrays (i.e., an array of a multiplicity of chips), microchips, microarrays, a microarray assembled on a single chip, or any other similar format.

The term “gene” refers to a polynucleotide sequence comprised of parts, that when operably combined in either a native or recombinant manner, provide some product or function. The term “gene” encompasses mRNA, cDNA, cRNA and genomic forms of a gene. In some but not all embodiments, genes comprise coding sequences necessary for the production of a polypeptide. In addition to the coding region of the polynucleotide, the term “gene” also encompasses the transcribed nucleotide sequences of the full-length mRNA adjacent to the 5′ and 3′ ends of the coding region are variable in size, and typically extend on both the 5′ and 3′ ends of the coding region. The sequences that are located 5′ and 3′ of the coding region and are contained on the mRNA are referred to as 5′ and 3′ untranslated sequences (5′ UT and 3′ UT, respectively).

As used herein, the term “regulatory element” refers to a genetic element which controls some aspect of the expression of polynucleotide sequences. For example, a promoter is a regulatory element that enables the initiation of transcription of an operably linked coding region. Other regulatory elements are splicing signals, polyadenylation signals, termination signals, etc. In some embodiments, the promoter sequence is “endogenous,” where the promoter is one which is naturally linked with a given gene in the genome. In other embodiments, the promoter is “exogenous,” or “heterologous,” where a non-natural promoter is placed in juxtaposition to a gene by means of genetic manipulation (i.e., molecular biological techniques such as cloning and recombination) such that transcription of the gene is controlled by the linked promoter.

The terms “in operable combination,” “in operable order,” “operably linked,” “operably joined” and similar phrases as used herein in reference to nucleic acids refer to polynucleotides that are placed in functional relationships with each other. For example, a promoter polynucleotide sequence and a gene open reading frame are operably linked when the combination results in accurate transcription of the gene to produce an RNA molecule.

As used herein, the term “gene expression” refers to the process of converting genetic information encoded in the genomic nucleotide sequence on a chromosome into RNA (e.g., mRNA, rRNA, tRNA, or snRNA) through “transcription” of the gene (i.e., via the enzymatic action of an RNA polymerase).

As used herein, the term “vector” is used in reference to polynucleotide molecules that transfer DNA segment(s) from one cell to another and are able to replicate in a suitable cell type. The term “vehicle” is sometimes used interchangeably with “vector.” A vector comprises parts which mediate its maintenance and enable its intended use (e.g., sequences necessary for replication, genes imparting drug or antibiotic resistance, a multiple cloning site, and operably linked promoter/enhancer elements which enable the expression of a cloned gene). Vectors are often derived from plasmids, bacteriophages, or plant or animal viruses. A “cloning vector” or “shuttle vector” or “subcloning vector” contains operably linked parts which facilitate subcloning steps (e.g., a multiple cloning site containing multiple restriction endonuclease sites).

The term “expression vector” as used herein refers to a vector comprising operably linked polynucleotide sequences necessary for the expression of an operably linked coding sequence in a particular host organism (e.g., a bacterial expression vector, a yeast expression vector or a mammalian expression vector). Polynucleotide sequences necessary for expression in prokaryotes typically include a promoter, an operator (optional), and a ribosome binding site, often along with other sequences. Eukaryotic cells utilize promoters, enhancers, and termination and polyadenylation signals and other sequences which are generally different from those used by prokaryotes.

The term “sample” as used herein is used in its broadest sense. The term “sample” as used herein is typically of biological origin, where “sample” refers to any type of material obtained from animals or plants (e.g., any fluid or tissue), cultured cells or tissues, cultures of microorganisms (prokaryotic or eukaryotic), and any fraction or products produced from a living (or once living) culture or cells. A sample can be unpurified or purified. A purified sample can contain principally one component, e.g., total cellular RNA, total cellular mRNA, cDNA or cRNA.

As used herein, the term “in vitro” refers to an artificial environment and to processes or reactions that occur within an artificial environment. The term “in vivo” refers to the natural environment (e.g., in an animal or in a cell) and to processes or reactions that occur within a natural environment. An in vitro transcription (IVT) reaction is a transcription reaction that takes place in a cell-free environment using largely purified components, e.g., purified DNA template and purified DNA-dependent RNA polymerase.

As used herein, the term “DNA-dependent DNA polymerase” refers to a DNA polymerase that uses deoxyribonucleic acid (DNA) as a template for the synthesis of a complementary and antiparallel DNA strand.

As used herein, the term “DNA-dependent RNA polymerase” refers to an RNA polymerase that uses deoxyribonucleic acid (DNA) as a template for the synthesis of an RNA strand. The process mediated by a DNA-dependent RNA polymerase is commonly referred to as “transcription.” Either strand in a double-stranded DNA molecule can be used as a template for RNA synthesis, and is dependent on the sequence and orientation of the RNA-polymerase promoter operably linked to the DNA molecule.

As used herein, the term “RNA-dependent DNA polymerase” refers to a DNA polymerase that uses ribonucleic acid (RNA) as a template for the synthesis of a complementary and antiparallel DNA strand. The process of generating a DNA copy of an RNA molecule is commonly termed “reverse transcription,” and the enzyme that accomplishes that is a “reverse transcriptase.” In some cases, an enzyme that demonstrates reverse transcriptase activity also demonstrates additional activities, such as but not limited to nuclease activity (e.g., RNaseH ribonuclease activity) and DNA-dependent DNA polymerase activity.

As used herein, the term “amplification” refers generally to any process that results in an increase in the amount of a molecule. As it applies to polynucleotide molecules, amplification means the production of multiple copies of a polynucleotide molecule, or part of a polynucleotide molecule, from one or few copies or small amounts of starting material. Amplification of polynucleotides encompasses a variety of chemical and enzymatic processes. The generation of multiple DNA copies from one or a few copies of a template DNA molecule during a polymerase chain reaction (PCR) is a form of amplification. Other amplification processes include strand displacement amplification (SDA; Beckton, Dickenson and Company, and Nanogen, Inc., San Diego, Calif.), transcription-mediated amplification (TMA; Gen-Probe®, Inc., San Diego, Calif.), and nucleic acid sequence-based amplification (NASBA; Organon-Teknika). Amplification is not limited to the strict duplication of the starting molecule. For example, the generation of multiple RNA molecules from a single DNA molecule during the process of transcription (e.g., in vitro transcription) is a form of amplification.

In some embodiments, amplification does not require any subsequent steps following the amplification reaction. In other embodiments, amplification is followed by additional steps, for example but not limited to, labeling, sequencing, purification, isolation, hybridization, expression, detecting and/or cloning.

As used herein, the term “polymerase chain reaction” (PCR) refers to a method for amplification well known in the art for increasing the concentration of a segment of a target polynucleotide in a sample, where the sample can be a single polynucleotide species, or multiple polynucleotides. Generally, the PCR process consists of introducing a molar excess of two or more extendable oligonucleotide primers to a reaction mixture comprising the desired target sequence(s), where the primers are complementary to opposite strands of the double stranded target sequence. The reaction mixture is subjected to a precise program of thermal cycling in the presence of a DNA polymerase, resulting in the amplification of the desired target sequence flanked by the DNA primers. Reverse transcriptase PCR (RT-PCR) is a PCR reaction that uses RNA template and a reverse transcriptase to first generate a single stranded DNA molecule prior to the multiple cycles of DNA-dependent DNA polymerase primer elongation. Multiplex PCR refers to PCR reactions that produce more than one amplified product in a single reaction, typically by the inclusion of more than two primers in a single reaction. Methods for a wide variety of PCR applications are widely known in the art, and described in many sources, for example, Ausubel et al. (eds.), Current Protocols in Molecular Biology, Section 15, John Wiley & Sons, Inc., New York (1994).

As used herein, the term “enrichment” refers to a change in relative proportion (i.e., percentage) of at least one species in a pool of multiple species, where the proportion of one or more species increases relative to another species. As used herein, amplification is not required to achieve enrichment. Furthermore, it is not a requirement that enrichment results in amplification. In some embodiments of the present invention, enrichment is optionally followed by an amplification step.

As used herein, the term “polymerase extension” refers to any template-dependent polymerization of a polynucleotide by any polymerase enzyme. The polymerase can be an RNA-dependent DNA polymerase (i.e., reverse transcriptase, e.g., Moloney murine leukemia virus [MMLV] reverse transcriptase), DNA-dependent RNA polymerase (e.g., T7 RNA polymerase), or a DNA-dependent DNA polymerase (e.g., Taq DNA polymerase or Bst DNA polymerase). Polymerase extension is not limited to polymerase activity that requires a primer to initiate polymerization. For example, T7 RNA polymerase does not require the presence of a primer for polymerase initiation and extension.

### DETAILED DESCRIPTION

One of the challenges to the quantitative and qualitative study of gene expression, as well as the isolation of certain genes, is the wide range of expression levels between different genes within a single cell or tissue. That is to say, the genes expressed in a given transcriptome show an unequal partitioning, where some genes are expressed at a significantly higher level than other genes. This range in gene expression levels is illustrated in a hypothetical example shown in TABLE 1.

TABLE 1

| Transcript | Transcript copies | Abundance in the |
|---|---|---|
| class | per cell | mRNA fraction |
| Low | <15 | <0.005% |
| Intermediate | 15-500 | 0.005-0.167% |
| High | >500 | >0.167% |

TABLE 1 provides one example of what can be considered low, intermediate or high levels of transcription. As can be seen in TABLE 1, the number of gene transcripts per cell (i.e., the copy number of the transcript) can vary by more than four orders of magnitude.

Furthermore, there is a disproportionately large number of genes represented in the low and intermediate classes of gene expression compared to a relatively small number of genes expressed at very high levels. This disparity results in relatively few high copy number gene transcripts accounting for approximately 10-20% of the mRNA population. In contrast, much larger numbers of intermediate abundance genes account only for 40-45% of the mRNA population, while the largest percentage of genes, the low abundance genes, represent 40-45% of the mRNA population.

As used herein, it is not intended that the terms “low” or “high” be rigidly defined in any respect. In one aspect, a gene or polynucleotide that is considered “highly transcribed” or “high abundance” (i.e., has a high copy number in the cell) has an abundance of at least 0.1% of the polynucleotides in a sample. For example, and without limitation, a high abundance gene may have 500 mRNA transcripts per every 300,000 mRNA transcripts (where 300,000 transcripts is an approximation of the number of mRNA molecules in any given cell at any given time), and thus, account for at least 0.167% of the polyA mRNA in a given cell, cell population or tissue. In a particular embodiment a “high abundance” polynucleotide represents at least about 0.2% of the polynucleotides in a sample. In another embodiment a high abundance polynucleotide represents at least about 0.5% of the polynucleotides in a sample. In a further embodiment a high abundance polynucleotide represents at least about 1% of the polynucleotides in a sample. In a still further embodiment a high abundance polynucleotide represents at least about 5% of the polynucleotides in a sample.

In another aspect, a gene or polynucleotide is considered to be “low abundance” if it has an abundance of less than about 0.1% of the polynucleotides in a sample. Thus, genes that are expressed at an intermediate level may be considered “low abundance.” In one embodiment “low abundance” polynucleotides represent less than about 0.05% of the polynucleotides in a sample. In another embodiment they represent less than about 0.01% of the polynucleotides in a sample. In a further embodiment they represent less than about 0.005% of the polynucleotides in a sample. In a still further embodiment, low abundance polynucleotides represent less than about 0.001% of the polynucleotides in a sample. For example, and without limitation, a gene that has a low level of transcription (i.e., has a low copy number in the cell) may have an abundance of not greater than 15 transcripts per every 300,000 mRNA transcripts, and thus, account for not more than 0.005% of the mRNA in a given cell, cell population or tissue.

The relationship of high abundance polynucleotides to low abundance nucleotides in a sample can be given as a ratio. In one embodiment a high abundance polynucleotide (or gene) is one that is expressed at a level at least about twice the level of expression of a low abundance polynucleotide. Thus, the ratio of the high abundance polynucleotide to the low abundance polynucleotide is at least about 2:1. In another embodiment a high abundance polynucleotide has an expression level at least about five times the expression level of a low abundance polynucleotide (10:1). In a further embodiment a high abundance polynucleotide has an expression level at least about ten times the expression level of a low abundance polynucleotide (100:1). In yet a further embodiment a high abundance polynucleotide has an expression level at least about fifty times the expression level of a low abundance polynucleotide (500:1). In a still further embodiment a high abundance polynucleotide has an expression level at least about one hundred time the expression level of a low abundance polynucleotide (1,000:1). In other embodiments the ratio of the high abundance polynucleotide to the low abundance polynucleotide may be at least about 5,000:1, 10,000:1 or greater.

The information in TABLE 1 has been demonstrated experimentally using various techniques, and is well documented in the art. For example, the unequal distribution of relative transcript abundance in the transcriptome has been demonstrated using real-time quantitative PCR analysis. Real-time PCR analysis refers to the periodic monitoring of accumulating PCR products (also known as a fluorogenic 5′ nuclease assay, i.e., TaqMan® analysis; see, Holland et al., Proc. Natl. Acad. Sci. USA 88:7276-7280 [1991]; and Heid et al., Genome Research 6:986-994 [1996]).

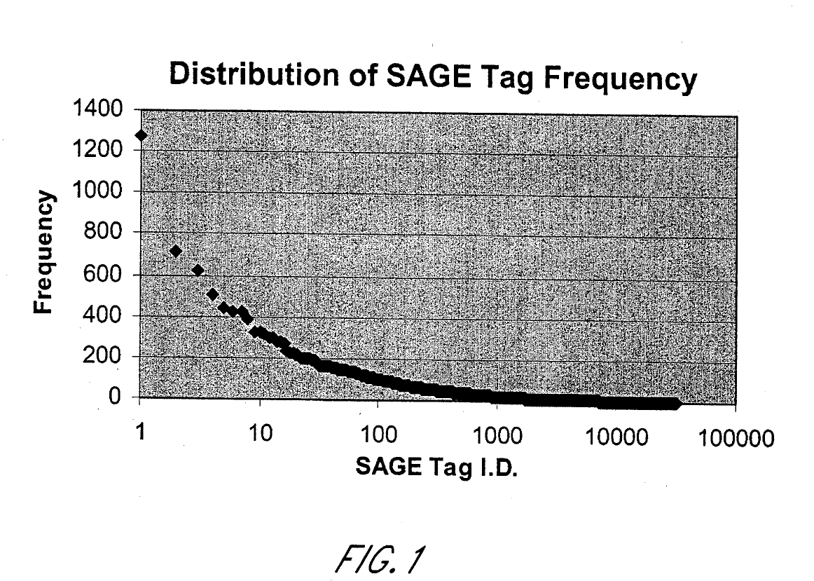

The unequal distribution of transcript distribution in living cells has also been demonstrated using serial analysis of gene expression (SAGE) analysis. The results of a publicly available SAGE analysis are shown in FIG. 1. SAGE is a method that takes advantage of high-throughput sequencing technology to provide quantitative analysis of cellular gene expression, without the need of providing an individual hybridization probe for each transcript analyzed.

Essentially, the SAGE technique measures not the expression level of a gene, but quantifies a “tag” that represents the transcription product of a gene. A tag, for the purposes of SAGE, is a nucleotide sequence of a defined length, typically about 9-14 basepairs in length, directly 3′ to the 3′-most restriction site for a particular restriction enzyme. The enzyme NlaIII remains the most widely used restriction enzyme, although other restriction enzymes can also be used. Many transcripts are linked together to form long serial molecules that can be rapidly sequenced, simultaneously revealing the identity of multiple tags. This approach has been used in SAGE tag-count sets in which roughly 250,000 total tags have been sequenced.

The expression pattern of any population of transcripts (i.e., the transcriptome) can be quantitatively evaluated by determining (i) the abundance of individual tags in the given transcriptome, and (ii) identifying the gene corresponding to each tag. The data product of the SAGE technique is a list of tags, with their corresponding count values, and thus is a digital representation of cellular gene expression. The methodologies and uses of SAGE analysis are known in the art, and are described in various sources. See, e.g., Velculescu et al., Science 270:484-487 (1995); Velculescu et al., Cell 88:243-251 (1997); and Zhang et al., Science 276:1268-1272 (1997).

As shown in the analysis in FIG. 1, the X-axis plots the SAGE Tag ID (10-mer oligonucleotides), and the Y-axis plots the frequency of appearance of a particular tag. The data set depicted in this graph is extracted from a publicly available database maintained by the National Center for Biotechnology Information at the National Institutes for Health. This analysis sampled 62,486 sequence tags from a cDNA library.

As can be seen in FIG. 1, a very small number of SAGE tags are represented in the transcriptome at a disproportionately high level. The vast majority of SAGE tags show moderate or low representation in the library. In fact, of the 62,486 tags sampled, many of them appeared only as single hits (i.e., represented only once in the sample). Conversely, a relatively small number of frequently appearing tags account for a majority of the tag hits in the sample.

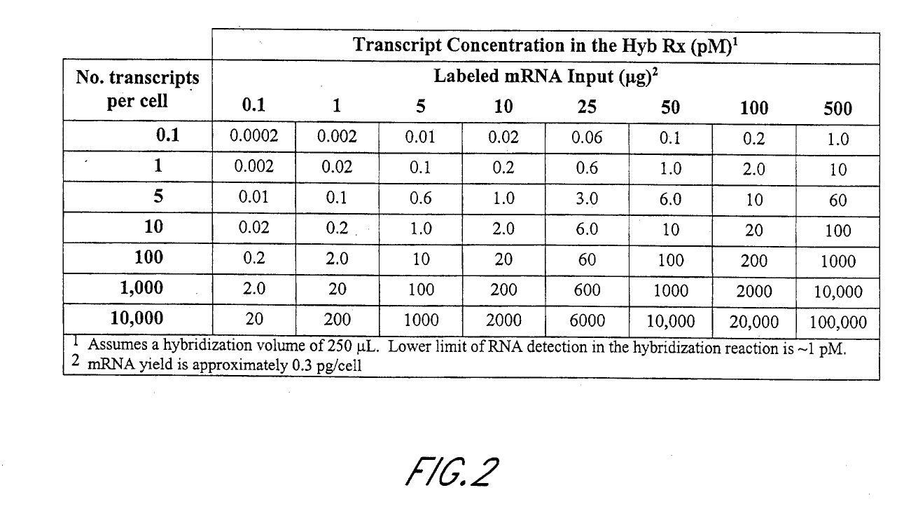

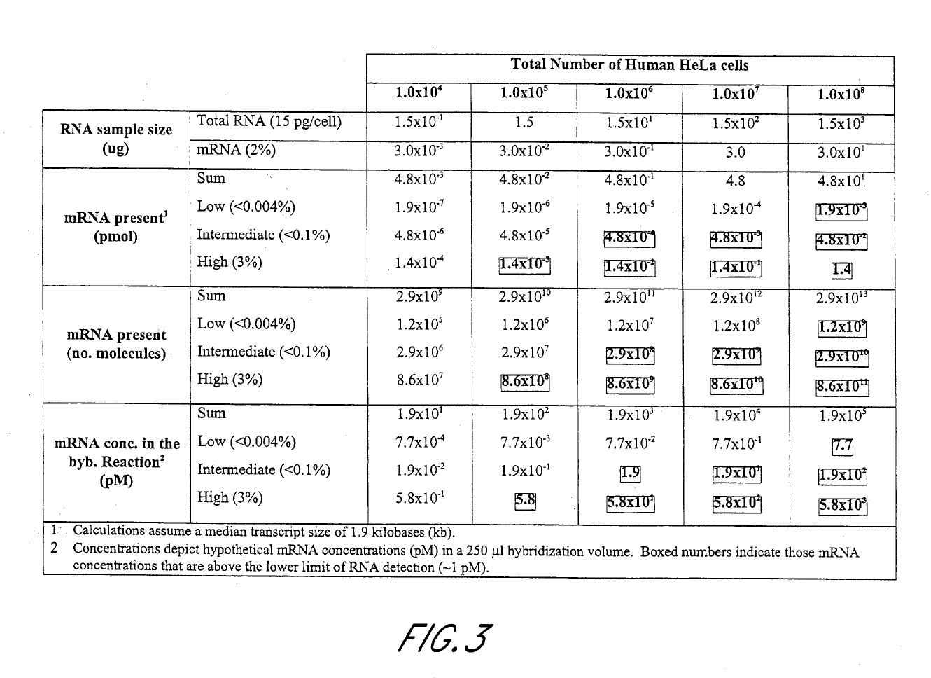

A hypothetical calculation of mRNA quantitation and concentration that illustrates limitations of the current art is shown in FIG. 2. In FIG. 2, the mRNA concentrations of seven different genes in a standard 250 μL hybridization reaction (typical of “chip” formats) is determined for eight different quantities of unamplified labeled mRNA input (0.1-500 μg). The genes shown in FIG. 2 represent a 100,000-fold range in expression levels. The predicted concentrations of each of the gene transcripts in the hybridization reaction are provided in pM. The lower limit of RNA detection in array formats is approximately 1 pM. Thus, any transcript in the table in FIG. 3 having a concentration lower than 1 pM would not be detectable. For example, if 5 μg of mRNA were used in the hybridization reaction, only transcripts having a copy number of 10 or greater would be detectable.

A similar example illustrating limitations in gene expression analysis is shown in FIG. 3. FIG. 3 shows a hypothetical calculation of mRNA quantitation given different amounts of mRNA starting material. The hypothetical RNA yield from 10⁴ through 10⁸ HeLa cells is calculated in μg, pmol and number of transcripts. This analysis assumes an average transcript length of 1.9 kilobases (kb), and makes these calculations for low, intermediate and high abundance classes of mRNA transcript. This analysis also determines the predicted mRNA molar concentration in a 250 μL hybridization reaction. Given a lower limit of detection of approximately 1 pM for a given mRNA (corresponding to a lower limit of detection for gene expression of approximately one transcript per cell in one million cells), gene transcripts above this detection limit are shown in boxes. Thus, starting with 10⁶ (one million) cells, only intermediate and high abundance mRNA transcripts can be detected.

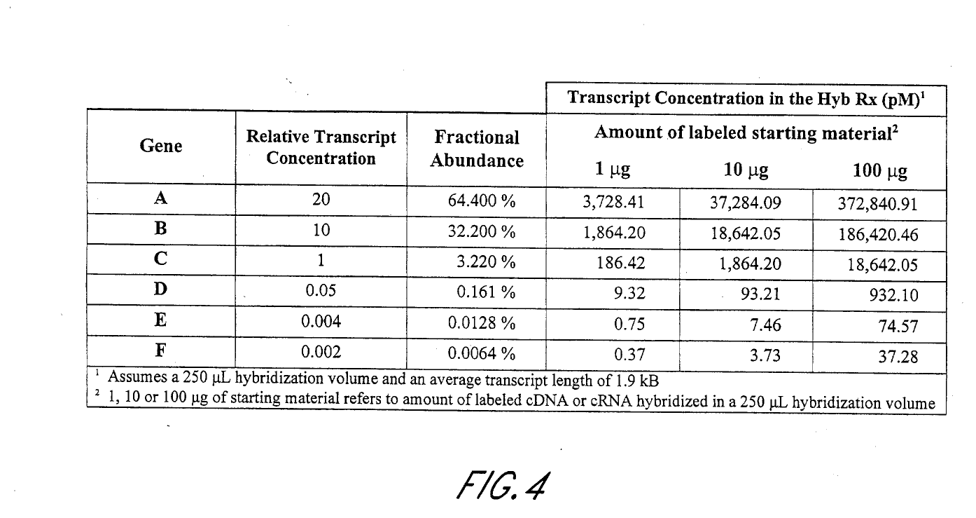

FIG. 4 also illustrates the difficulty in analyzing low-abundance transcripts. Similar to FIGS. 2 and 3, FIG. 4 provides hypothetical calculations of polynucleotide (cDNA or cRNA) concentrations in a hybridization reaction, where six different genes having a 10,000-fold difference in expression level (genes A-F) are analyzed using three different amounts of starting material. Again, these calculations show that the lowest abundance transcripts are not detectable using currently known methods that can analyze only small quantities of starting material.

Increasing the amount of polynucleotide starting material (either unamplified mRNA or total RNA, amplified cRNA, cDNA or sense or antisense IVT product) in a hybridization analysis could compensate for the problem of low levels of gene expression. However, there is a practical limitation to the amount of polynucleotide that can be used in a hybridization reaction. Using standard laboratory conditions, there is a practical upper limit to the amount of amplified RNA that can be generated by an in vitro transcription (IVT) labeling reaction (approximately 100 μg). In addition, highly expressed genes or transcripts will consume a large portion of IVT reagents and thus reduce the yield of low-expressed, targeted genes. There is also a practical limitation to the amount of mRNA (i.e., polyA RNA) that can be generated and labeled for analysis, as mRNA accounts for only 1-5% of the total cellular RNA. Another concern is the potential for probe cross hybridization caused by the extremely high concentrations of the highest abundance transcripts.

From the calculations in FIGS. 2-4, it is apparent that the current art is hindered by poor detection and analysis of low-abundance polynucleotides (e.g., primary mRNA transcripts, cDNA molecules or cRNA) using the microarray hybridization format. Thus, there is a need in the art for compositions and methods for the improved detection and analysis of low-abundance polynucleotides. Furthermore, there is a need in the art for compositions and methods that specifically enrich or selectively amplify low abundance transcripts, such that the low-copy number transcripts can be detected and/or analyzed using any variety of techniques presently known in the art for the analysis of polynucleotides or gene expression.

Presently used methods for the selective removal of targeted polynucleotides in a sample suffer from technical limitations. Some of these methods use subtractive hybridization (i.e., hybridization-based pull-out) to capture and remove targeted sequences. Other methods use specific enzymatic degradation (e.g., RNaseH digestion) to remove transcripts that have formed duplexes with defined oligonucleotides. These methods are suboptimal due to poor yield, requirement for large amounts of starting material, and non-specific loss/degradation of desired low-abundance polynucleotides. These approaches frequently fail to identify low-abundance species in a sample of polynucleotides.

A. Enrichment Of Low-Abundance Polynucleotides

One way to avoid the need to increase the total amount of starting material used for the analysis of low-abundance polynucleotides (i.e., mRNA transcripts) is to enrich the polynucleotide sample for the low-abundance species. This approach provides advantages over simply increasing the amount of analysis material used in a hybridization reaction. First, this approach eliminates the potential for non-specific cross hybridization of abundant messages to the hybridization probes, which would result in false positive results. Second, it results in an increase of the relative abundance of the moderate and low abundance messages. This means that for a given amount of material used in a hybridization reaction or other application, each of the remaining sequences is present in a higher proportion and will therefore be more easily detected, quantified and/or isolated.

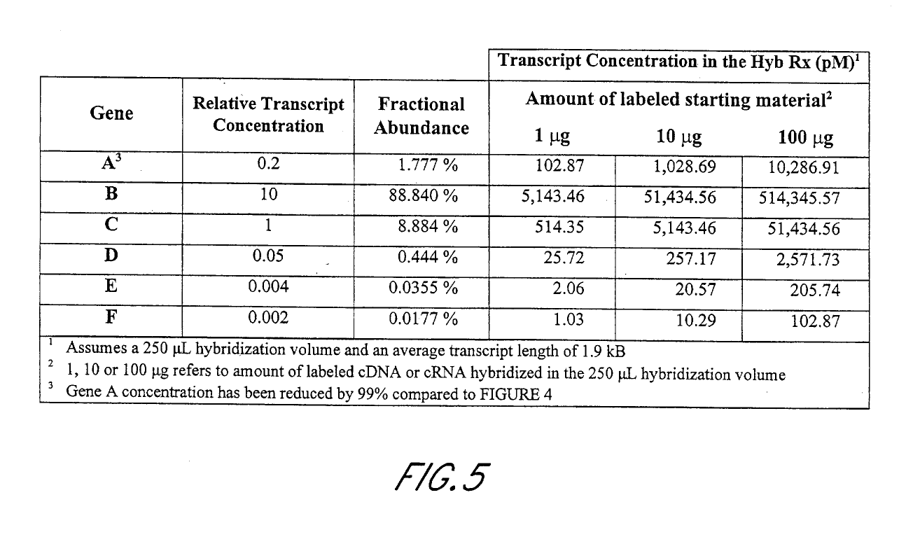

Enrichment for low-abundance species in a sample can be accomplished by the selective reduction of the most abundant species in the sample. This principle is demonstrated in a simple hypothetical scenario provided in FIGS. 4 and 5, illustrating what occurs to relative transcript concentrations upon amplification of six different genes (genes A-F). FIG. 4 shows a hypothetical analysis of gene expression, where six different genes (genes A-F) having a 10,000-fold range in levels of expression are amplified (as either cDNA or cRNA molecules) and analyzed in a hybridization method. Three scenarios are provided, where 1, 10 or 100 μg of labeled material (i.e., cDNA or cRNA) are used in the hybridization reactions. The predicted concentrations of each of the gene transcripts in the hybridization reaction are provided in pM. As can be seen in these calculations, when using 1 μg of starting material, the lower-abundance transcripts (i.e., genes E and F) are not detectable, as they have concentrations below 1 pM. In this case, 10 μg of labeled material must be hybridized in order to detect the lowest expressed transcript (i.e., gene F). In the more complex case of human mRNA, the amount of material required to detect transcripts having even lower levels of expression is expected to be higher.

The calculations made in FIG. 5 are analogous to those made in FIG. 4, except that the level of the most abundant transcript (i.e., gene A) has been reduced by 99%. As can be seen in FIG. 5, when the level of gene A is decreased, the fractional abundance of the other transcripts increases to detectable levels. Therefore, by selectively blocking the amplification of certain species, a relative enrichment of other species is observed, and this approach can overcome the limits of non-selective amplification alone, as depicted in FIG. 4.

B. Novel Compositions and Methods for the Enrichment of Low Abundance Polynucleotides

The present invention provides compositions and methods for the enrichment of low abundance polynucleotides in a sample. These methods enrich a sample for low abundance species by exposing the polynucleotides in a sample to conditions for enzymatic polymerization, and simultaneously suppressing the polymerization of at least one high abundance species in the sample. The inhibition of polymerization of at least one abundant polynucleotide species results in the relative enrichment of other less abundant species in the sample (as demonstrated in the hypothetical examples in FIGS. 4 and 5).

These novel methods combine the polymerization of desired species (i.e., low or moderate abundant species) and the suppression of polymerization of non-desired species (i.e., at least one high abundance species) in a single reaction, and thus simplifies the enrichment process. By combining these two steps into a single step, loss and/or degradation of sample, especially low abundance or rare species in a sample, is minimized. The methods of the invention do not require large amounts of starting material (e.g., especially mRNA), and thus, find particular use in the analysis of samples where the amount of starting material is limited. The compositions and methods of the present invention find use in a variety of applications, as detailed below.

Furthermore, following the enrichment, the polynucleotide sample can optionally be used in any of a variety of amplification steps as known in the art. These amplification mechanisms include PCR, in vitro transcription, or subcloning with plasmid/phagemid expansion.

The methods of the present invention yield polynucleotide pools that are enriched in low abundance polynucleotide species compared to the starting polynucleotide pool, and thus, facilitate the detection and/or isolation of low abundance species (e.g., mRNA or cRNA transcripts, or cDNA molecules). These novel methods utilize sequence-specific non-extendable nucleobase oligomers that preferentially block the polymerization of high-abundance target molecules in a pool of molecules, and thus, increase the relative proportion of low abundance transcripts. These blocking oligomers are added to the sample prior to initiating a polymerase amplification reaction. The blocking oligomers anneal to their target sequence and create a duplex that selectively suppresses the amplification of the target polynucleotide in the pool of polynucleotides by blocking the progression or initiation of a polymerase enzyme, i.e., primer extension. Thus, the methods of the present invention do not require any specialized equipment or other instrumentation.

One or multiple low abundance polynucleotides may be amplified in the polymerization process. They may be amplified individually through the use of primers that are specific for the sequence of each low abundance polynucleotide to be amplified. Alternatively, all polynucleotides in the sample, other than those that are blocked, may be amplified through methods well known in the art, such as by using random primers (see, e.g., Feinberg, A. P. and Vogelstein, B. 1983. Analyt. Biochem. 132:6-13; Feignberg, A. P. and Vogelstein, B. 1984. Analyt. Biochem. 137:266-267). A random primer comprises a mixture of all possible permutations of a given n-mer, where n is, for example, 6, 7, 8, 9 or 10. Typically, random hexamers (n=6) or octamers (n=8) are employed. However, it may be possible to amplify all polynucleotides in a sample, other than the high-abundance polynucleotides that are to be blocked, using a subset of all possible permuations of a given n-mers. Thus, in one embodiment a random primer comprises 100 distinct n-mers (i.e. 100 hexamers, each with a distinct sequence). In another embodiment a random primer comprises 200 distinct n-mers. In still further embodiments a random primer may comprise 400, 800, 1000 or 5000 different n-mers. Random primers can be purchased commercially, or prepared using an oligonucleotide synthesizer (Applied Biosystems, Foster City, Calif.).

The methods of the invention can be applied to any situation where a low-abundance polynucleotide is in a sample of polynucleotides, where more abundant polynucleotides prevent or hinder the detection or isolation of the low-abundance species. This sequence-specific suppression of high-abundance species, and consequent enrichment of low-abundance species, permits the detection, isolation and/or analysis of the low-abundance polynucleotides that were previously too low in concentration to be detected or isolated prior to the enrichment. In some embodiments, the invention provides methods for labeling a pool of polynucleotides that have been enriched in low-abundance transcripts, where the labeled pool of polynucleotides finds use, for example, in methods for the analysis of gene expression or gene cloning. In other embodiments, the invention provides kits that facilitate the present methods, where the kits provide various reagents to use in the methods.

The methods of the present invention utilize blocking nucleobase oligomers that are enzymatically non-extendable. It is not intended that the chemical structure of the non-extendable nucleobase oligomers be particularly limited, except where the oligomer retains the ability to hybridize to a complementary target in a sequence-specific manner. A variety of non-extendable nucleobase structures are known in the art, all of which find use with the invention. The oligomers are designed to be complementary to an abundant (i.e., highly transcribed) target sequence in the sample, and are hybridized to the target.

In some embodiments, more than one blocking oligomer is used in the polymerase reaction, and thus, the polymerization of more than one high abundance polynucleotide is simultaneously blocked.

It is not intended that the site of duplex formation between the blocking oligomer and target molecule be particularly limited. In some embodiments, a site of duplex formation that is more proximal to the site of polymerase initiation is preferable over a site of duplex formation that is more distal from the site of polymerase initiation. In other embodiments, the site of duplex formation overlaps or encompasses the polymerase start site.

C. Methods for the Enrichment of Low Abundance mRNA Molecules

In some embodiments, the present invention provides novel methods to suppress the DNA polymerization of at least one abundant mRNA in a sample, where the mRNA is converted to the first strand of a complementary DNA (cDNA) molecule by an RNA-dependent DNA polymerase activity (i.e., reverse transcriptase; RT). This is accomplished by the inclusion of novel blocking oligomers in the RT reaction, where the oligomers are complementary to one or more abundant mRNA transcripts in the sample. These blocking oligomers form duplexes that block the initiation or extension of a first strand cDNA product from an oligo-dT primer, and thus result in failure of the reverse transcriptase enzyme to initiate first strand cDNA synthesis, or prevent the generation of a full length first strand of the cDNA.

In other embodiments, blocking oligomers are present in the cDNA second strand synthesis reaction, where the blocking oligomers are complementary to the newly synthesized first strand of DNA that may have escaped the blockage during the first strand synthesis. The blocking oligomers used in this embodiment hybridize to the opposite strand that is targeted in the first strand synthesis reaction. These blocking oligomers specific for the second strand have nucleobase sequences that are distinct from the nucleobase oligomer sequences used to block the generation of the cDNA first strand. The regions targeted for duplex formation with the blocking oligomer(s) in the first cDNA strand may or may not be different from the regions targeted for duplex formation with the blocking oligomer(s) in the second cDNA strand.

It is contemplated that blocking oligomers can be used either during the cDNA first strand synthesis, during the cDNA second strand synthesis, or in both the first and second strand synthesis reactions. In the case where the blocking oligomers are used in both the first and second strand cDNA synthesis (without an intervening purification step), the blocking oligomers used in the two enzymatic steps are designed to hybridize to different regions of the target gene in order to prevent formation of non-productive oligomer/oligomer duplexes.

In some embodiments of the invention, the cDNA second strand is synthesized by a DNA-dependent DNA-polymerase activity and primed by random DNA oligomers. However, it is not intended that the present invention be limited to this one method for second strand synthesis, as alternative protocols for second strand cDNA synthesis are known to one of skill in the art, and which find use with the present invention.

This modified RT reaction generates a pool of double-stranded complementary DNAs (cDNAs) that is enriched in cDNAs derived from low abundance transcripts as compared to a pool of RT reaction products that would be generated without the use of the blocking oligomers. This biased cDNA pool generated by the novel methods of the present invention have a variety of uses, including, but not limited to, microchip array hybridization (i.e., gene expression analysis), use in in vitro transcription (IVT) reactions to generate cRNA products, cDNA library synthesis and screening, SAGE analysis, and other applications.

D. Enrichment of Polynucleotide Sequences Using PCR

In other embodiments, blocking nucleobase oligomers can be incorporated directly in a PCR reaction. In this case, the blocking oligomers can target either one or both strands of a double-stranded DNA template molecule (e.g., a double-stranded cDNA). In one embodiment of this method, the Tₘ of the blocking oligomer(s) is preferably higher than the Tₘ of the primers used in the PCR reaction.

In the case where blocking oligomers specific for both strands of the double-stranded DNA template are used simultaneously, the two blocking oligomers have nucleobase sequences that are distinct from each other, and furthermore, the blocking oligomers used are designed to hybridize to different regions of the double stranded target in order to prevent formation of non-productive oligomer/oligomer duplexes through complementary base-pairing.

The inclusion of the blocking oligomers in the PCR reaction results in the failure or reduced ability to generate PCR amplicons containing the targeted sequence. For example, this application finds use in blocking the PCR amplification of known high abundance sequences during the amplification of a cDNA library, such as when the cDNA library is cloned into a vector that permits the use of universal primers for PCR amplification of the entire library.

E. Methods for the Generation of RNA Enriched in Low Abundance Species by In Vitro Transcription

As described above, the invention provides novel methods for the generation of a population of cDNA molecules that have been enriched for low abundance species as a consequence of suppressing the polymerization of at least one high abundance species. In some embodiments, the cDNA molecules thus-formed can be operably linked with a nucleotide sequence suitable for the initiation of transcription, i.e., in vitro transcription (IVT), using a DNA-dependent RNA-polymerase (e.g., T7 RNA polymerase). Thus, the cDNA pool can be used as template material in an IVT reaction to generate a pool of RNA enriched in low abundance species.

IVT reactions are, in general, amplification reactions, as they produce large amounts of RNA from minimal starting quantities of a DNA template. The DNA template can be amplified up to 1000-fold in an IVT reaction. IVT reactions utilize a DNA template (e.g., a cDNA molecule or pool of cDNA molecules) having an operably linked promoter initiation sequence, a DNA-dependent RNA polymerase (e.g., T7, SP6 or T3 RNA polymerases) and free ribonucleotide triphosphates (rNTPs) to enzymatically produce RNA molecules complementary to one strand of the starting DNA template.

The double-stranded cDNA IVT template is generally a linear molecule. The cDNA molecule can consist primarily of a cDNA sequence operably linked to the transcription promoter, or alternatively, the cDNA can be subcloned into a suitable vector (e.g., a bacteriophage ?-based vector, e.g., ?-gt11 or ?-gt12, or a circularized expression vector). In some embodiments, the circularized vector containing the cDNA is linearized prior to the IVT reaction.

In these methods, the DNA-dependent RNA-polymerase can be used to generate either an antisense transcript (i.e., complementary, or cRNA) or a “sense” RNA transcript. A sense RNA transcript is a transcript that is produced in the same orientation as its corresponding endogenous transcript. That is, the sense transcript has the same orientation and the same, or substantially the same, nucleotide sequence as the primary mRNA transcript. In contrast, a cRNA has a sequence that is complementary to the corresponding mRNA product. Whether a sense or antisense product is formed is dependent on the orientation of transcription.

A wide variety of reagents and reaction conditions for performing IVT are known in the art, and which find use with the present invention. It is not intended that the present invention be limited to the IVT reaction conditions and reagents specifically recited herein, as these conditions are only exemplary in nature. Methods and reagents for IVT are common in the art and are available from various manufacturers, and are described in many sources, for example, Ausubel et al. (eds.), Current Protocols in Molecular Biology, Vol. 1-4, John Wiley & Sons, Inc., New York (1994) and Sambrook et al. (eds.), Molecular Cloning: A Laboratory Manual, Second Edition, Vol. 1-3, Cold Spring Harbor Laboratory Press, NY, (1989).

The RNA products generated by the IVT reaction find use in a variety of applications, including, but not limited to, microchip array hybridization in the analysis of gene expression, and other applications. In one embodiment, the IVT RNA products are labeled during their synthesis for use in the hybridization analysis (see, EXAMPLE 3).

F. Demonstration of Various Embodiments of the Invention

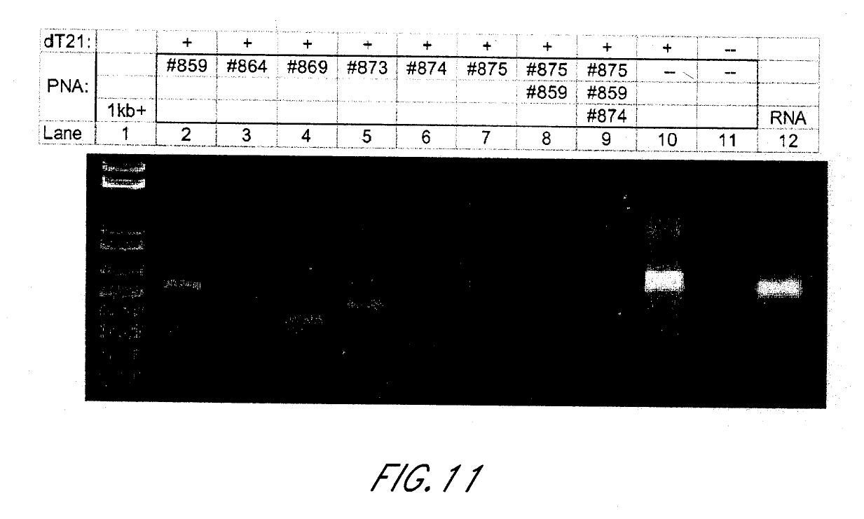

Various properties and advantages of the invention were demonstrated in a series of experiments, shown in FIGS. 11-13. In these experiments, non-extendable nucleobase oligomers were designed to bind an mRNA target sequence to form duplexes that impede reverse transcriptase enzyme from transcribing the target sequence and generating the first strand of a complementary DNA sequence (i.e., cDNA first strand synthesis). In this one case, non-extendable peptide nucleic acid (PNA) oligomers were used as the blocking oligomer. The synthesis of PNA oligomers and hybridization properties of PNA oligomers are known in the art (Buchardt et al., WO 92/20702; Nielsen et al., Science 254:1497-1500 [1991]; Egholm et al., Nature 365:566-568 [1993]).

The PNA oligomers used herein are intended to be exemplary for the purpose of illustrating various properties of the invention. It is not intended that the invention be limited to the nucleobase sequences used herein, nor be limited to the use of molecules having PNA structures. As discussed elsewhere, a variety of additional blocking oligomer sequences and structures find use with the invention, and it is intended that the broadest aspects of the invention encompass such alternative reagents. Furthermore, it is not intended that the present invention be limited to the reverse transcriptase reagents and reaction conditions specifically recited herein, as one familiar with the art will recognize that equivalent conditions also find use with the invention.

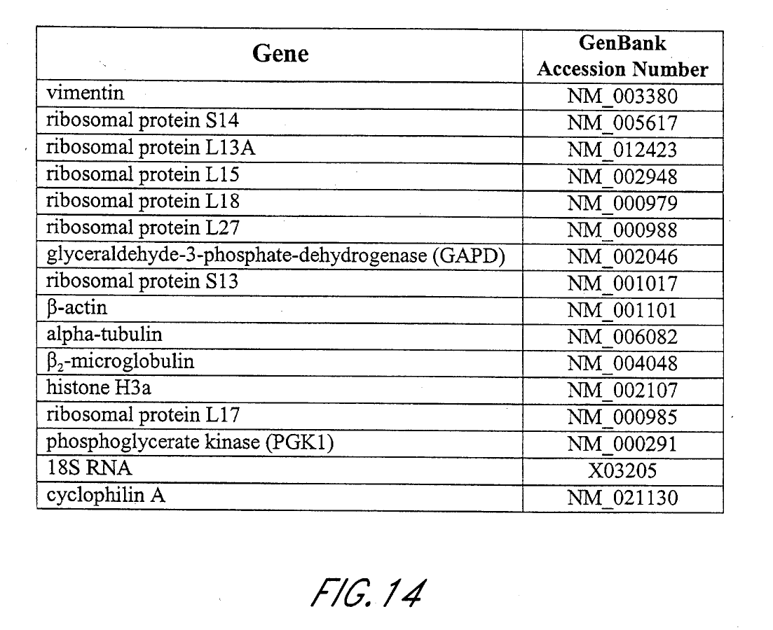

The PNA oligomers were designed to be complementary to two different gene transcripts, which were the human import precursor of subunit B of the H⁺ transporting, mitochondrial ATP synthase, subunit B, isoform 1 gene (ATP5F1; GenBank Accession Number NM_{—}001688) and the cholesteryl ester transfer protein gene (CETP; GenBank Accession Number NM_{—}000078). The ATP5F1 and CETP gene sequences were used herein in an exemplary manner to illustrate various properties of the invention. It is not intended that the invention be limited to the use of blocking oligomers specific for these target genes. As discussed elsewhere, nucleobase sequences specific for a variety of additional target genes also find use with the invention and are encompassed by the broadest aspects of the invention. A list of additional highly expressed genes finding use as blocking targets is shown in FIG. 14.

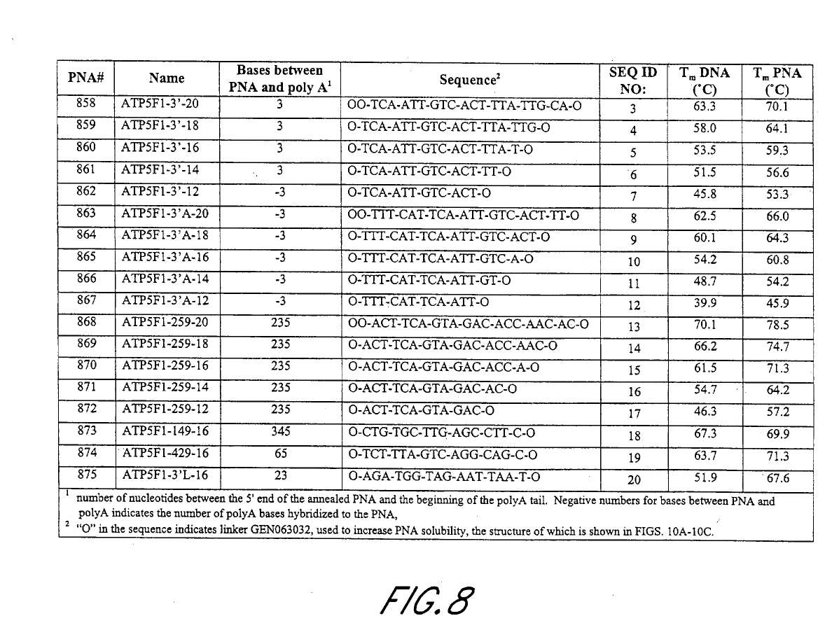

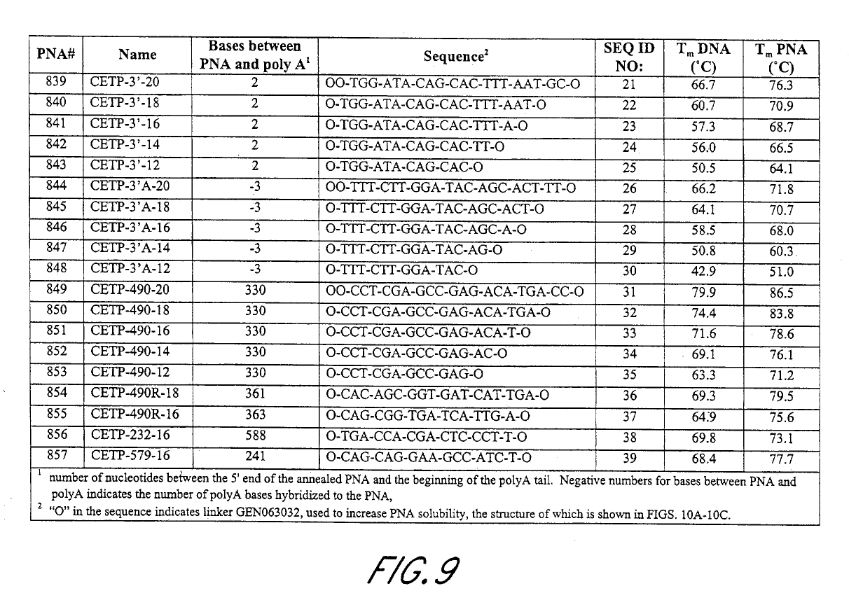

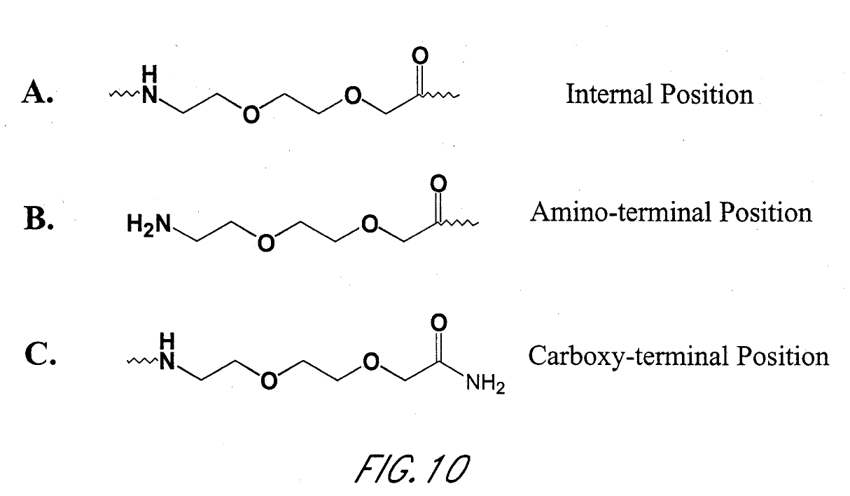

Synthetic transcripts of truncated versions of the ATP5F1 and CETP genes were used in these polymerase reactions. PNA oligomers were designed and synthesized to bind to several different regions of each transcript, including overlapping the first 3 A's of the polyA tail, 3 bases upstream from the polyA tail, and other sites internal to the gene. The PNA nucleobase sequences of these oligomers specific for the ATP5F1 and CETP genes are provided in FIGS. 8 and 9, respectively, and are also provided in SEQ ID NOs: 3-20, and 21-39, respectively. As used in FIGS. 8 and 9, the “O” character in the PNA sequences indicates a linker/spacer moiety, termed GEN063032 (Applied Biosystems, Foster City, Calif.), incorporated to improve the solubility of the PNA oligomer, as known in the art (see, WO 99/37670; and Gildea et al., Tetrahedron Letters 39:7255-7258 [1998]). The structure of this linker/spacer is shown in FIGS. 10A-10C. FIG. 10A shows this structure when the linker is at an internal position in the oligomer. FIG. 10B shows the structure of the linker when it is in the amino-terminal position. FIG. 10C shows the structure of the linker when it is in the carboxy-terminal position.

Oligomers varying in length and duplex melting temperature (Tₘ) were tested in order to determine whether an optimal PNA oligomer to block polymerase activity could be identified. As shown in FIGS. 8 and 9, the calculated Tₘ of the PNA oligomer and the analogous DNA oligomer are shown for comparison. The Tₘ of the PNA oligomer is uniformly higher than the corresponding DNA oligomer, indicating that the PNA-containing heteroduplex is more stable and energetically favorable than the analogous DNA duplex.

Reverse transcription reactions using a recombinant MMLV reverse transcriptase (GibcoBRL® SUPERSCRIPT II™ reverse transcriptase), an artificial ATP5F1 transcript, an oligo-dT₂₁ RT primer, and several different PNA oligomers were used to demonstrate the ability of the PNA oligomers to inhibit a reverse transcription reaction in a target-specific manner.

The results of this analysis are shown in FIG. 11. Single-stranded cDNA products of the RT reactions were resolved on an agarose gel, and detected using ethidium bromide staining. Lane 12 shows 60 ng of the 626 ribonucleotide template for size comparison, and lane 10 shows the reverse transcribed single-stranded 573 deoxyribonucleotide product in the absence of any PNA oligomers, revealing a single predominant product of approximately the same size as the template. Lane 11 is a control reaction that omits the oligo-dT primer. The inhibitory effect of the various PNA oligomers can be clearly observed. PNA numbers 859 and 864 are the same length and have the same predicted Tₘ, however, 864, which binds the first 3 bases of the polyA tail, appears to have a stronger blocking effect. Reactions with PNA numbers 869 and 873, which bind 235 and 345 nucleotides, respectively, from the polyA tail appear to produce small amounts of cDNA of approximately those sizes. Lanes 8 and 9 demonstrate that using two or three PNA sequences in concert in a single RT reaction further improves RT blocking efficiency, where no cDNA product was detectable in these reactions.

The results provided in FIG. 11 indicate that all PNA oligomers specific to the ATP5F1 transcript that were tested (numbers 859, 864, 869, and 873-875) showed some ability to suppress reverse transcription and production of cDNA product, and thus, all find use with the invention.

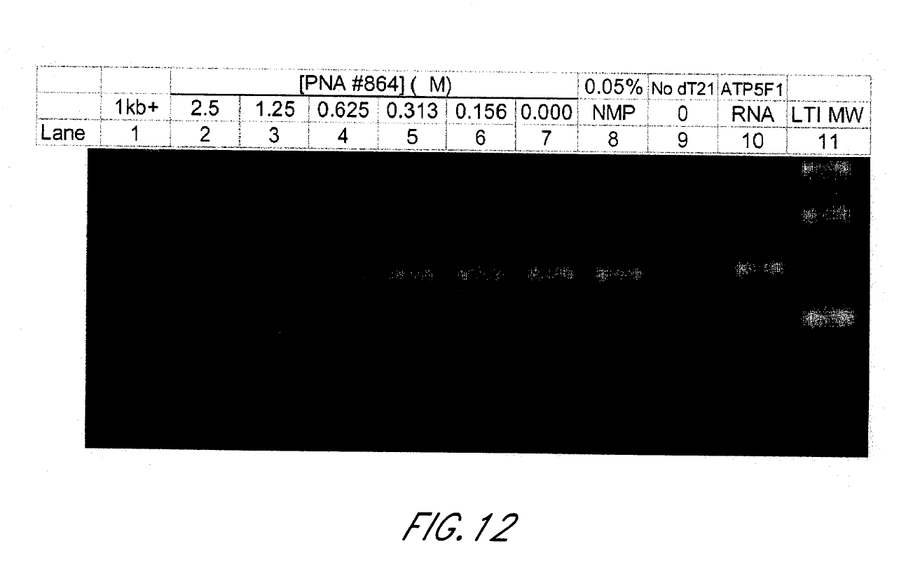

In order to demonstrate that this inhibitory effect was due to RT blocking by the PNA oligomers, various control experiments were performed using the ATP5F1 transcript template. The results of these experiments are shown in FIG. 12. In FIG. 12, lane 10 shows the ribonucleotide template, lane 7 shows the reverse transcribed single-stranded DNA product in the absence of PNA oligomers, and lane 9 is a control reaction that omits the oligo-dT primer. Lanes 1 and 11 show DNA size markers. It was tested whether the solvent used to dissolve the PNAs (1% N-methylpyrrolidone [NMP]) by itself was able to inhibit the RT reaction. As can be seen in FIG. 12, lane 8, 0.05% NMP in the RT reaction had no effect on RT activity and the generation of a single-stranded cDNA product.

It was also tested whether the RT inhibition observed was dependent on the dose of PNA oligomer. FIG. 12, lanes 2-7, show the effects of a range of PNA concentrations in the RT reaction products. PNA oligonucleotide number 864 was used in two-fold dilutions. In these reactions, the molar concentration of the ATP5F1 transcript template was 0.4 μM. When the PNA concentration is raised above 0.4 μM, inhibition is observed, suggesting a one-to-one stoichiometry of PNA binding to its target.

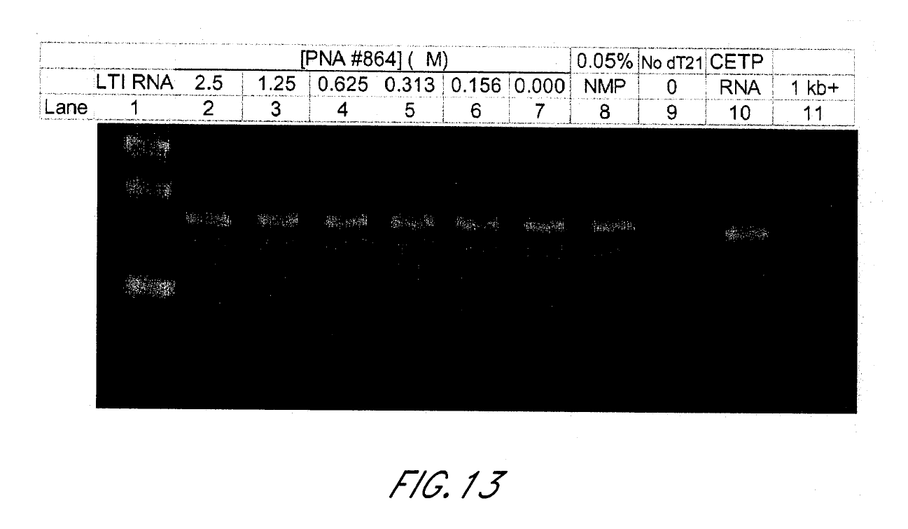

In order to demonstrate the sequence specificity of the blocking activity, the same ATP5F1 PNA oligomer dilution series was used in a series of RT reactions with a heterologous template, the CETP gene. The results of this experiment are shown in FIG. 13. In these reactions the final concentration of CETP transcript template was 0.3 μM. Even at the highest concentration of ATP5F1-specific PNA oligomer (2.5 μM), there is no inhibition of the CETP RT reaction, indicating that the blocking is highly sequence-specific and not due to non-specific interference.

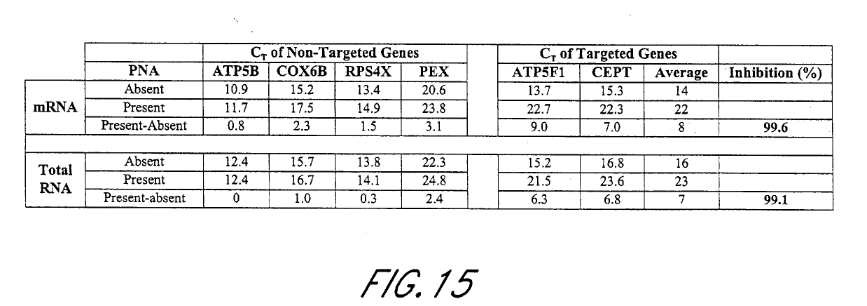

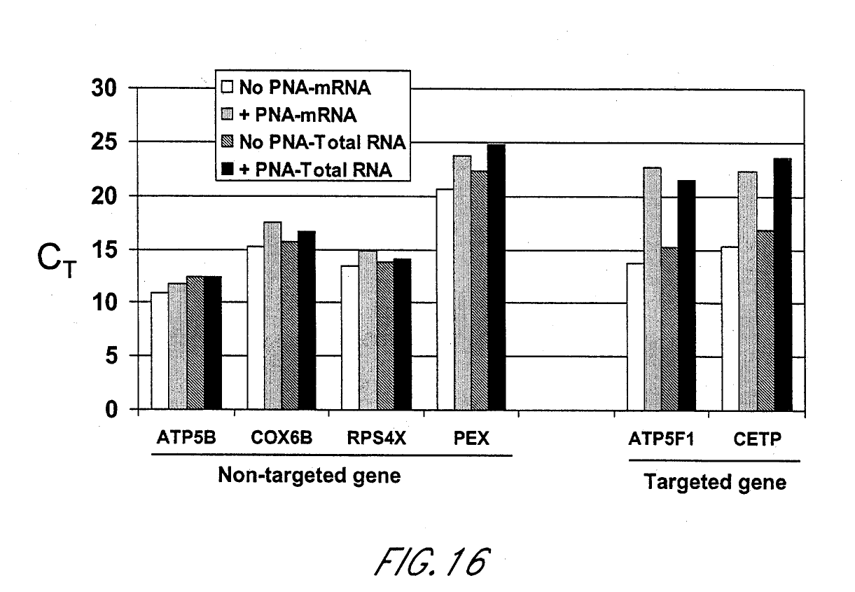

In a separate set of experiments using RNA isolated from human liver tissue, the ability of non-extendable oligomers to block the reverse transcription of targeted transcripts in samples of total cellular RNA and mRNA isolated from human cells was demonstrated. In these experiments, unlabeled cRNA products produced from an in vitro transcription reaction (as described in EXAMPLE 3) were quantitated using a TaqMan® RT-as PCR protocol (as described in EXAMPLE 4), as commonly used in the art. The effectiveness of the blocking oligomers to block the generation of cDNA molecules corresponding to various transcripts in the RNA samples in the reverse transcriptase step was assessed. The results of the analysis are shown in FIGS. 15-16.

Real-time quantitative PCR analysis (also known as a fluorogenic 5′ nuclease assay, i.e., TaqMan® analysis; see, Holland et al., Proc. Natl. Acad. Sci. USA 88:7276-7280 [1991]; and Heid et al., Genome Research 6:986-994 [1996]) refers to the periodic monitoring of accumulating PCR products.

In the TaqMan® PCR procedue, two oligonucleotide primers are used to generate an amplicon typical of a PCR reaction. A third oligonucleotide (the TaqMan® probe) is designed to detect nucleotide sequence located between the two PCR primers. The probe has a structure that is non-extendible by Taq DNA polymerase enzyme, and is labeled with a reporter fluorescent dye and a quencher fluorescent dye. The laser-induced emission from the reporter dye is quenched by the quenching dye when the two dyes are located close together, as they are on the probe.

The TaqMan® PCR reaction uses a thermostable DNA-dependent DNA polymerase that retains a 5′-3′ nuclease activity, such as Taq DNA polymerase. During the PCR amplification reaction, the Taq DNA polymerase cleaves the labeled probe that is hybridized to the amplicon in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data, such that the amount of released fluorescent reporter dye is directly proportional to the amount of starting amplicon template.

TaqMan® RT-PCR can be performed using commercially available equipment, such as, for example, ABI PRISM® 7700 Sequence Detection System (Applied Biosystems. Foster City, Calif.), or Lightcycler (Roche Molecular Biochemicals, Mannheim, Germany). In a preferred embodiment, the 5′ nuclease procedure is run on a real-time quantitative PCR device such as the ABI PRISM® 7700 Sequence Detection System. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

TaqMan® assay data are expressed as the threshold cycle (C_{T}). As discussed above, fluorescence values are recorded during every PCR cycle and represent the amount of product amplified to that point in the amplification reaction. The PCR cycle when the fluorescent signal is first recorded as statistically significant is the threshold cycle (C_{T}).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g., Heid et al., Genome Research 6:986-994 (1996).

In the present case, cRNA generated following RT and IVT amplification (see EXAMPLE 3) was used in a real-time PCR quantitation assay using a TaqMan® protocol. The cRNA products from the two targeted genes, ATP5F1 and CETP (as described in EXAMPLE 2), were quantitated. In addition, the cRNA products from four non-targeted genes was also assayed. These non-targeted genes were ATP5B (Homo sapiens ATP synthase, H⁺ transporting, mitochondrial F1 complex, β polypeptide; GenBank Accession No. NM_{—}001686), COX6B (Homo sapiens mitochondrial cytochrome c oxidase subunit VIb; GenBank Accession No. NM_{—}001863), RPS4X (Homo sapiens X-linked ribosomal protein S4; GenBank Accession No. NM_{—}001007), and PEX7 (Homo sapiens peroxisomal biogenesis factor 7; GenBank Accession No. NM_{—}000288). Quantitation was by RT-PCR using the cRNA as template, coupled with TaqMan® analysis (see EXAMPLE 4).

The results of this TaqMan® analysis are shown in FIG. 15. Results are expressed as C_{T}, or the threshold cycle, defined as the PCR cycle number where the detectable fluorescent signal from the TaqMan® probe is first recorded as statistically significant. C_{T} values are converted to actual concentrations by calibration against a stardardization curve (data not shown). This analysis revealed that PNA oligomers can effectively block the transcription of specific target genes (ATP5F1 and CEPT) by 99.1 and 99.6% during RT using either mRNA or total cellular RNA starting material as template, respectively. Furthermore, these data also demonstrate that these same blocking PNA oligomers used to inhibit the ATP5F1 and CEPT reverse transcriptase reactions do not inhibit the reverse transcription of non-targeted genes (i.e., ATP5B, COX6B, RPS4X and PEX7). This data is shown in FIG. 15 is also shown graphically in FIG. 16.

G. High Copy Number Gene Transcripts

It is widely recognized that the transcriptome of any given cell is not equally partitioned among all the expressed genes. On the contrary, it is recognized that relatively few genes account for the vast majority of mRNA transcripts found in any given cell. Such genes are known as “high copy number” genes, as transcripts of these genes are disproportionately abundant in the cellular mRNA pool.

It is contemplated that such high copy number gene transcripts can be targeted by blocking oligomers in methods of the present invention to block their polymerization and amplification. For example, a non-extendable nucleobase oligomer complementary to an abundant gene transcript can be utilized during first strand cDNA synthesis (i.e., a reverse transcriptase reaction) to suppress the DNA-polymerization of the abundant transcripts into cDNA from an mRNA sample. In some embodiments, a single high-abundance polynucleotide is targeted with the blocking oligomer. In other embodiments, more than one high-abundance species is simultaneously targeted with blocking oligomers. Furthermore, as different cell types display different patterns of expressed genes, it is contemplated that different blocking oligomers or combinations of oligomers are optimally used in the enrichment of low abundance polynucleotides from various samples.

It is not intended that the blocking oligomers of the present invention be limited to targeting the ATP5F1 or CETP genes. On the contrary, a large number of high abundance (i.e., high copy-number) genes are known. Examples of high-abundance genes are provided in a non-exhaustive list of FIG. 14, along with the respective GenBank Accession Numbers for the gene cDNA sequences. The genes listed in this figure are exemplary only, as additional high-abundance genes (i.e., mRNAs) are widely known in the art. Furthermore, abundant ribosomal RNA's (e.g., 18S and 28S rRNA species) are also suitable targets for blocking oligomers, as used in the methods of the present invention.

Furthermore, amplification of more than one high abundance polynucleotide may be blocked simultaneously, such as by the use of the appropriate number of specific blocking oligomers. In one embodiment at least 2 high abundance polynucleotides are blocked. In another embodiment at least about 5 high abundance polynucleotides are blocked. In a further embodiment at least 10 high abundance polynucleotides are blocked. In a still further embodiment from at least 50 to at least 100 high abundance polynucleotides are blocked. In other embodiments at least 20, 30, 40, 50, 75, 100, 250, 500, 1000 or more high abundance polynucleotides are blocked.

Similarly, more than one low abundance polynucleotide can be amplified simultaneously. For example, in one embodiment the amplification process amplifies all polypeptides that are not blocked. In another embodiment at least 2 low abundance polynucleotides are amplified. In yet another embodiment at least five low abundance polynucleotides are amplified. In a further embodiment at least 10 low abundance polynucleotides are amplified. In other embodiments at least 20, 30, 40, 50, 75, 100, 250, 500, 1000 or more low abundance polynucleotides are amplified.

In another aspect, one or more high abundance polynucleotides are blocked while one or more low abundance polynucleotides are amplified. In one embodiment at least 2 high abundance polynucleotides are blocked while at least 2 low abundance polynucleotides are amplified. In another embodiment at least 5 high abundance polynucleotides are amplified while at least 10 low abundance polynucleotides are amplified. In another embodiment at least 20 high abundance polynucleotides are blocked while at least 20 low abundance polynucleotides are amplified. In a further embodiment at least 20 high abundance polynucleotides are blocked while at least 40 low abundance polynucleotides are amplified. In other embodiments at least 20, 30, 40, 50, 75, 100, 250, 500, 1000 or more low abundance polynucleotides are amplified while at least 20, 30, 40, 50, 75, 100, 250, 500, 1000 or more high abundance polynucleotides are blocked.

H. Source and Isolation of RNA for Use in the Reverse Transcription Reaction

It is not intended that the source of RNA template to be used in a reverse transcriptase reaction to generate cDNA products be limited to any particular source. Non-limiting examples of sources of RNA include tissues, whole blood or cultured cells, and furthermore, can be obtained from any organism. In some embodiments, RNA is derived from human tissues, human blood, or cultured human cells. RNA can be used with the present invention as a pool of total cellular RNA, or as polyA RNA (i.e., the RNA sample is predominantly mRNA having 3′-polyadenylation). RNA that is available from commercial sources also finds use with the present invention.

The method used to isolate RNA used in the present invention is not limited to any particular method or methods. Methods for total RNA and poly-A RNA isolation are common in the art, and are described in various sources (See, e.g., Ausubel et al. (eds.), Current Protocols in Molecular Biology, Section 4, Part I, John Wiley & Sons, Inc., New York [1994]; and Sambrook et al. (eds.), Molecular Cloning: A Laboratory Manual, Second Edition, Chapter 7, Cold Spring Harbor Laboratory Press, NY, [1989]). Non-limiting examples of RNA isolation methods which find use with the invention include guanidium isothiocyanate lysis with cesium chloride gradient sedimentation and differential precipitation. Furthermore, methods for RNA isolation using commercially available products are common in the art, and include, for example, QIAGEN® RNeasy® total RNA isolation kits and QIAGEN® Oligotex® polyA RNA isolation kits.

I. Reverse Transcriptase Reactions

The present invention provides methods whereby RNA is reverse transcribed to form the first strand of a cDNA molecule (reverse transcription) in the presence of an RNA-dependent DNA-polymerase (reverse transcriptase) enzyme. A wide variety of reverse transcriptase reaction conditions and reagents are well known in the art, and it is not intended that the present invention be limited to the specific RT reaction conditions or reagents recited in this application. Various equivalent RT reaction conditions can be found in sources such as Ausubel et al. (eds.), Current Protocols in Molecular Biology, Vol. 1-4, John Wiley & Sons, Inc., New York (1994) and Sambrook et al. (eds.), Molecular Cloning: A Laboratory Manual, Second Edition, Vol. 1-3, Cold Spring Harbor Laboratory Press, NY, (1989).

The reverse transcriptase enzyme used with the invention need not have RNaseH activity. Thus, reverse transcriptase enzymes with or without RNaseH activity find use with the present invention. Reverse transcriptase enzymes from any organism or virus find use with the invention, including but not limited to, for example, recombinant forms of Moloney murine leukemia virus (MMLV or MoMuLV) reverse transcriptase and avian myeloblastosis virus (AMV) reverse transcriptase. Reverse transcriptase enzymes are readily available from commercial sources, including for example, Stratagene®, Promega®, Invitrogen™, GibcoBRL®, QIAGEN®, Roche™ Biochemicals and Sigma®/Aldrich®.

It is also not intended that the invention be limited to any particular reverse transcriptase primer used for first strand cDNA synthesis. As described herein, the first strand cDNA synthesmay be primer is an oligo-dT based primer. Other types of RT primers, for example, template specific primers or random hexamer primers also find use with the invention.

It is not intended that the method for cDNA second strand synthesis of the invention be limited to any particular method. As described herein, cDNA second strand synthesis may be initiated using random priming. However, one familiar with the art knows other equivalent methods, which are encompassed by the present invention. For example, second strand cDNA synthesis can be accomplished by (i) intrinsic DNA-dependent DNA polymerase activity of the reverse transcriptase enzyme, or (ii) addition of RNaseH to nick the RNA template to produce 5′-RNA ends suitable for priming DNA synthesis by a suitable DNA ploymerase.

In addition, the polymerase primer can be engineered to comprise additional advantageous nucleotide sequences. For example, as described above, the primer sequence can comprise the promoter recognition sequence for bacteriophage T7 DNA-dependent dependent RNA polymerase. This minimal T7 promoter recognition sequence is:

5′-AATACGACTCACTATAG-3′ (SEQ ID NO: 40) Similarly, the bacteriophage SP6 and T3 promoter sequences also find use with the invention, as these promoter sequences can similarly promote in vitro transcription using SP6 or T3 DNA-dependent RNA polymerases, respectively. These sequences are known in the art.

Also, the RT primer can include still other sequence suitable for use as target sequences for PCR primers (i.e., universal PCR primer sequences) to facilitate subsequent PCR amplification. Restriction enzyme recognition sequences can also be engineered into the reverse transcriptase primer, so that useful restriction sites appear in the double-stranded cDNA product, which facilitates cDNA subcloning, if desired.

DNA restriction enzymes, subcloning techniques, and other molecular genetic techniques are common in the art, and are described in numerous sources. Similarly, reagents for use in such protocols are readily available from a large number of commercial vendors.

endable, Blocking Nucleobase Oligomers

Certain nucleobase oligomers comprising various modified nucleotide bases, nucleotide analogs or modified chain backbones are unable to serve as primers (i.e., are enzymatically non-extendable) in the initiation of enzymatic DNA or RNA synthesis by DNA-dependent or RNA-dependent polymerases. A large number of these structures are know in the art, and are described in various sources (see, e.g., WO 95/08556 and WO 99/34014). As used herein, non-extendable oligomers of the invention refer to oligomers that bind to either RNA or DNA, or more typically, can bind to both RNA and DNA; i.e., the non-extendable extendable oligomers of the invention have blocking activity for both RNA-dependent polymerases and DNA-dependent polymerases. While the nucleobase oligomer sequences are able to bind complementary polynucleotide molecules in a sequence-specific manner, enzymatic DNA or RNA synthesis (i.e., initiation or extension) does not occur due to the non-extendable chemical structure of the nucleobase oligomer. For example, some oligomers are unable to be enzymatically extended because they lack a 3′ hydroxyl group on the ribose sugar ring required for nucleotide addition.

A large number of non-extendable modified nucleotides and other nucleobase structures find use with the present invention, and it is not intended that methods of the invention be limited to the use of any one particular non-extendable nucleobase structure. However, various properties of the nucleobase oligomers make some species more preferable than other species. These preferred characteristics are, 1) oligomers of defined base sequence can be readily synthesized and have some solubility in aqueous solution, 2) the oligomers are able to bind complementary polynucleotide sequences in a sequence-specific manner to form stable heteroduplexes, 3) the heteroduplexes are not subject to nuclease digestion, and 4) the blocking oligomer is a non-extendable primer substrate for DNA polymerase or RNA polymerase (i.e., can not initiate nucleotide chain elongation). In other embodiments, it is preferable that the Tₘ of the blocking oligomer is higher than the Tₘ of an oligonucleotide primer used to initiate nucleic acid synthesis from the same template.

Non-limiting examples of non-extendable nucleobase oligomer structures known in the art and that find use with the invention are discussed below.

Peptide (or polyamide) nucleic acids, also known as PNAs, find use with the invention as blocking oligomers. PNAs are nucleobase oligomeric molecules where the phospho-diester ribose backbone of a polynucleotide has been replaced by an achiral, acyclic uncharged pseudopeptide backbone composed of repeating polyamide structural units. The PNA backbone forms a scaffold for covalently attached nucleobases to form oligomeric structures having defined base sequences. A PNA backbone composed of repeating N-(2-aminoethyl)glycine units are used in the present invention; however, it is not intended that the PNA structures of the invention be limited to this structure. Alternative PNA structures and methods for the synthesis of PNA oligomers are known in the art (Hyrup and Nielsen, Bioorg. Med. Chem., 4(1):5-23 (1996); WO 92/20702 and WO 92/20703). PNA oligomers can be synthesized using tBoc or Fmoc solid phase synthesis, and custom oligomer sequences can be readily ordered from commercial services (e.g., Applied Biosystems, Foster City, Calif.).

These PNA molecules share some properties with nucleotide oligomers, but also have significant differences. First, PNA oligomers are able to hybridize with RNA or DNA to form stable heteroduplexes, and these heteroduplexes have a greater Tₘ than do duplexes of oligodeoxyribonucleotides having the same base sequence. Second, PNA oligomers can not serve as primers to initiate enzymatic chain elongation for reverse transcriptase or any other DNA or RNA polymerase enzyme, and furthermore, PNA oligomers have the ability to block nucleotide chain elongation when hybridized downstream in a polynucleotide template. Third, PNA-containing duplexes are not a substrate for RNascH cleavage or cleavage by other nuclease activities encoded by polymerase enzymes. Also, as shown in FIG. 11, the length of the PNA oligomer or position of hybridization do not appear to be particularly limiting in order to display polymerase blocking activity.

In some embodiments, the PNA oligomers additionally and optionally comprise a linker/spacer moiety, termed GEN063032 (Applied Biosystems, Foster City, Calif.), incorporated to improve the solubility of the PNA oligomer, as known in the art (see, WO 99/37670; and Gildea et al., Tetrahedron Letters 39:7255-7258 [1998]). This linker/spacer can be incorporated in an internal, amino-terminal, or carboxy-terminal position, and one or more than one linker/spacer can be incorporated into the oligomer. The structure of this linker/spacer in these various positions is shown in FIGS. 10A-10C.

In other embodiments, the PNA molecules used in the invention are chiral molecules, i.e., have enantiomeric forms. Peptide nucleic acids having chiral structures are known in the art (D'Costa et al., Tetrahedron Letters 43:883-886 [2002]).

In alternative embodiments, other oligomeric nucleobase structures find use with the invention. The synthesis and properties of these structures are described in the art. These structures include locked nucleic acids (LNAs; see, WO 98/22489; WO 98/39352; and WO 99/14226), 2′-O-alkyl oligonucleotides (e.g., 2′-O-methyl modified oligonucleotides; see Majlessi et al., Nucleic Acids Research, 26(9):2224-2229 [1998]), 3′ modified oligodeoxyribonucleotides, N3′-P5′ phosphoramidate (NP) oligomers, MGB-oligonucleotides (minor groove binder-linked oligs), phosphorothioate (PS) oligomers, C₁-C₄ alkylphosphonate oligomers (e.g., methyl phosphonate (MP) oligomers), phosphoramidates, β-phosphodiester oligonucleotides, and a-phosphodiester oligonucleotides.

It is further contemplated that blocking oligomers of the present invention can be chimeric in structure, where the oligomer comprises two or more portions of differing chemical structure (see, e.g., U.S. Pat. No. 6,316,230). As with uniform oligomeric structures (e.g., PNA oligomers), the chimeric oligomers of the invention may be enzymatically non-extendable, and block the initiation or elongation of transcription of the polynucleotide to which it is specifically hybridized.

K. Subcloning of Double-Stranded cDNA Products and cDNA Library Construction

In other embodiments of the present invention, the cDNA products that have been enriched in low abundance species are subcloned into vectors to allow other applications. A pool of subcloned products forms a cDNA “library.” A subcloned cDNA pool permits the propagation of these cDNA molecules without the necessity of reproducing the reverse transcriptase reaction that created them. This is significant where extremely limited quantities of mRNA starting material are available, and where the cDNA products will be used in a variety of applications.

For example, the creation of cDNA libraries that have been enriched in low-abundance transcripts is a valuable embodiment of the present invention, especially in view of some genes which have been intractable to cloning efforts due to the low-copy number and scarcity of the gene mRNA. Also, a cDNA pool can be subcloned into a vector that permits forward or reverse transcription, where transcription in the forward direction produces sense transcripts suitable for translation and expression screening.

Methods for the manipulation of recombinant DNA molecules, cloning techniques and suitable vectors, including plasmid and viral (e.g., phage) vectors, are common in the art, and are described in many sources, for example, Ausubel et al. (eds.), Current Protocols in Molecular Biology, Vol. 1-4, John Wiley & Sons, Inc., New York (1994) and Sambrook et al (eds.), Molecular Cloning: A Laboratory Manual, Second Edition, Vol. 1-3, Cold Spring Harbor Laboratory Press, NY, (1989).

L. Applications

The present invention finds use with a variety of protocols. For example, the compositions and methods of the invention find use in the analysis of gene expression, and in cDNA library construction. However, it is not intended that the invention find use in only these applications. Indeed, one familiar with the art will immediately recognize a variety of uses for methods that enrich for low abundance polynucleotides in a sample. Similarly, the pools of enriched polynucleotides created by using the novel methods also find a variety of uses. The uses cited herein are intended to be exemplary, and such examples are not exhaustive.

1) Analysis of Gene Expression

The cDNA and cRNA products provided by the present invention find use in hybridization assays in the analysis of gene expression. In this embodiment, polynucleotide samples that have been enriched in low-abundance polynucleotides are used in hybridization reactions to detect gene expression, and especially, in the detection of low copy number genes. The polynucleotide pools enriched in low-abundance species and amplified, as provided by the present invention, allow the detection of low copy-number species, where previously the low copy-number species were undetectable by methods currently used in the art.

In some embodiments, the hybridization reactions take place in high throughput formats, as known in the art. It is not intended that the present invention be limited to any particular hybridization format or protocol, as one familiar with the art is familiar with a variety of hybridization protocols, and recognizes well the advantages of the present invention as they apply to many high throughput screening formats.

Generally, the high throughput hybridization formats use a probe that is affixed to a solid support. The solid support can be any composition and configuration, and includes organic and inorganic supports, and can comprise beads, spheres, particles, granules, planar or non-planar surfaces, and/or in the form of wells, dishes, plates, slides, wafers or any other kind of support. In some embodiments, the structure and configuration of the solid support is designed to facilitate robotic automation technology. The steps of detecting, measuring and/or quantitating can also be done using automation technology.

In some embodiments, the hybridization format is an “array”, “microarray”, “chip” or “biochip” as widely known in the art (see, e.g., Ausubel et al. (eds.), Current Protocols in Molecular Biology, Chapter 22, “Nucleic Acid Arrays,” John Wiley & Sons, Inc., New York [1994]; and M. Schena, (ed.), Microarray Biochip Technology, BioTechnique Books, Eaton Publishing, Natick, Mass. [2000]). In general, array formats facilitate automated analysis of large numbers of samples and/or have a large number of addressable locations, so that patterns of gene expression for a very large number of genes can be studied very rapidly. It is contemplated that a large number of array formats find use with the present invention, and it is not intended that the present invention be limited to any particular array format.

The use of polynucleotide pools enriched in low abundance species in hybridization assays typically necessitates the labeling of the polynucleotide pool prior to hybridization. A variety of labeling techniques are known in the art, and it is not intended that the present invention be limited to any particular polynucleotide labeling method. As used herein, “label” refers to any moiety that allows detection or visualization, but which by itself may or may not be detectable (e.g., fluorescein or biotin, respectively). A label that by itself is not detectable becomes detectable by its interaction with secondary molecule(s), e.g., strepavidin coupled to a fluorescent dye. The labeled polynucleotides permit the detection of those species that are in a duplex with a probe affixed to a solid support, such as in a microarray. A labeled polynucleotide in the duplex with the affixed probe can be detected using a variety of suitable methods, which can encompass colorimetric determinations, fluorescence, chemiluminescence and bioluminescence.

In one embodiment of the invention, the labeling of the polynucleotide pool (comprising either RNA or DNA molecules) is accomplished by incorporating a suitable label into the nascent polynucleotide molecules at the time of synthesis. For example, as described herein, dye-coupled UTP can be incorporated into a nascent RNA chain (see, EXAMPLE 3)

In an alternative embodiment, the labeling of the polynucleotide pool is accomplished after the polynucleotide pool is synthesized. In these embodiments, the RNA or DNA molecules are labeled using a suitable label that is coupled (i.e., conjugated or otherwise covalently attached) to the polynucleotides after chain synthesis.

In still other embodiments, the unlabeled pool of polynucleotides enriched for low abundance species produced by the present invention can be used directly in hybridization or gene expression analysis using methods that do not required a labeling step. For example, duplex formation with an affixed probe can be detected using surface plasmon resonance (SPR). See, e.g., Spreeta™ SPR biosensor (Texas Instruments, Dallas, Tex.), and BIACORE® 2000 (BIACORE®, Uppsala, Sweden). Resonant light scattering methods can also be used to detect duplex formation in a hybridization analysis using probes that have not been otherwise labeled (Lú et al., Sensors 1:148-160 [2001]).

It is not intended that the present invention be limited to any particular labeling method. One skilled in the art is familiar with a wide variety of alternative labeling protocols and reagents, all of which find use with the present invention.

2) cDNA Library Synthesis and Screening

Methods provided by the present invention can be used to generate pools of cDNA that are enriched in low-abundance transcripts. In one embodiment, these cDNA pools can be used to create cDNA libraries enriched for low abundance messages, where these libraries find use in the identification and isolation of genes represented by low copy number mRNA molecules. In other embodiments, these cDNA pools that are enriched for low-abundance species can also be used to directly sequence a rare species directly from the cDNA pool (either before or after the construction of a cDNA library).

Methods for the creation of cDNA libraries following the generation of cDNA molecules are known in the art. Similarly, methods for cDNA library screening are also widely known, and include, for example, homology screening and DNA/protein interaction screens, and various forms of expression screening such as antibody-based immunoscreening, protein/protein interaction screening, and screenings based on functional assays. Methods and reagents for library construction and screening are available in a variety of sources, including but not limited to, Ausubel et al. (eds.), Current Protocols in Molecular Biology, Vol. 1-4, John Wiley & Sons, Inc., New York (1994) and Sambrook et al. (eds.), Molecular Cloning: A Laboratory Manual, Second Edition, Vol. 1-3, Cold Spring Harbor Laboratory Press, NY (1989).

3) Cross Hybridization (i.e., Non-Specific Hybridization) Testing

The compositions and methods provided by the present invention find use in assays for determining the sequence specificity of a particular probe. For example, it is frequently desirable to determine the specificity of a probe for a particular nucleotide sequence contained in a mixed sample of many polynucleotide sequences (e.g., in total cellular RNA or in mRNA). That is to say, it is advantageous to learn if a probe will hybridize only to a target sequence, or if the probe will hybridize to other sequences in addition to the intended target that are contained in the sample (i.e., does the probe show non-specific cross hybridization). This is accomplished by comparing hybridization signals achieved using two different polynucleotide samples, where one sample is the “wild-type” sample containing all species, and the second sample is a “test” sample devoid of the target sequence.

Previously, this type of information has only been available in cases where there is a gene deletion (e.g., a knock-out) mutation, such as can be prepared in experimental organisms. As this type of experiment can not be done in human systems, this type of information as it applies to humans has been previously unavailable. However, the compositions and methods of the present invention provide pools of polynucleotides that have been specifically depleted for a single species of polynucleotide. Thus, these pools can be used in hybridization signal testing to determine the specificity of a probe to hybridize to a specific target in a human sample or a sample of any other organism.

M. Articles of Manufacture

The present invention provides articles of manufacture. Most significantly, the invention provides pools of polynucleotides that have been enriched for low-abundance species. These enriched polynucleotide samples can be in the form of cDNA molecules, or more typically, are in the form of cDNA libraries, where the cDNA molecules have been cloned into a plasmid, phagemid, or some other suitable vector. These cDNA libraries can optionally be in the form of an expression library, where the cDNA is cloned into a suitable vector that permits the transcription and translation of the cloned sequences. Enriched cDNA libraries can be prepared from any species, tissue or cell line. The cDNA libraries can be packaged in suitable containers, such as tubes or ampules that can be chilled or frozen during shipping and/or storage.

The invention also provides kits to facilitate the methods of the present invention, i.e., methods for the generation of pools of polynucleotides that are enriched for low-abundance species by the use of blocking nucleobase oligomers. Materials and reagents to carry out these methods can be provided in kits to facilitate execution of the methods.

As used herein, the term “kit” is used in reference to a combination of articles that facilitate a process, method, assay, analysis or manipulation of a sample. Kits can contain chemical reagents or enzymes required for the method, as well as other components. In some embodiments, the present invention provides kits for reverse transcription of cellular mRNA. These kits can include, for example but not limited to, reagents for the harvesting and/or collection of cells or tissues, reagents for the collection and purification of mRNA, a reverse transcriptase, primer suitable for reverse transcriptase initiation and first strand cDNA synthesis, at least one suitable blocking nucleobase oligomer, primer suitable for second strand cDNA synthesis, a DNA-dependent DNA polymerase, free deoxyribonucleotide triphosphates, and reagents suitable for the isolation/purification of the cDNA molecules produced by the reaction.

In other embodiments, the present invention provides kits for in vitro transcription of cDNA molecules and the production of cRNA. These kits can include, for example but not limited to, a DNA-dependent RNA polymerase, at least one suitable blocking nucleobase oligomer, free ribonucleotide triphosphates, and reagents suitable for the isolation/purification of the cRNA molecules produced by the reaction.

In one embodiment providing kits of the invention, blocking nucleobase oligomers are provided that are specific for a single high copy number gene. In other embodiments, blocking nucleobase oligomers specific for a plurality of target genes are provided. In one embodiment at least 2 high abundance polynucleotide blocking oligomers are provided. In another embodiment at least about 5 high abundance polynucleotide blocking oligomers are provided. In a further embodiment at least 10 high abundance polynucleotide blocking oligomers are provided. In a still further embodiment from at least 50 to at least 100 high abundance polynucleotide blocking oligomers are provided. In other embodiments at least 20, 30, 40, 50, 75, 100, 250, 500, 1000 or more high abundance polynucleotide blocking oligomers are provided.

The plurality of blocking oligomers provided in the kits may or may not be used simultaneously in a single polymerase reaction. Furthermore, the blocking nucleobase oligomers provided in the kits of the invention can be optimized for use in various cell types, where the blocking oligomers are specific for target sequences known to be highly expressed in the specific cell type under study. For example, in the study of gene expression in epithelial cells, it could be advantageous to block the amplification of highly expressed keratin genes in order to facilitate the detection or isolation of less abundant transcripts.

The kit may also include primers for the amplification of one or more low abundance polynucleotides. For example, the kit may comprise one or more random primers for the amplification of all polynucleotides, as described above. The kit may also comprise one or more primers that are specifically designed for the amplification of a particular low abundance polynucleotide. For example, in one embodiment the kit comprises primers for the specific amplification of at least 2 low abundance polynucleotides. In yet another embodiment the kit comprises primers for the specific amplification of at least five low abundance polynucleotides. In a further embodiment the kit comprises primers for the specific amplification of at least 10 low abundance polynucleotides. In other embodiments the kit comprises primers for amplifying at least 20, 30, 40, 50, 75, 100, 250, 500, 1000 or more low abundance polynucleotides.

The kit may comprise one or more blocking oligomers for blocking the amplification of high abundance genes as well as one or more primers for the amplification of low abundance polynucleotides. In one embodiment blocking oligomers are provided for blocking at least 2 high abundance polynucleotides and primers are provided for amplifying at least 2 low abundance polynucleotides. In another embodiment blocking oligomers are provided for blocking at least 5 high abundance polynucleotides and primers are provided for amplifying at least 10 low abundance polynucleotides. In yet another embodiment blocking oligomers are provided for blocking at least 20 high abundance polynucleotides and primers are provided for amplifying at least 20 low abundance polynucleotides. In a further embodiment blocking oligomers are provided for blocking at least 20 high abundance polynucleotides and primers are provided for amplifying at least 40 low abundance polynucleotides. In a still further embodiment, blocking oligomers are provided for blocking from 1 to 50 high abundance polynucleotides and a random primer is provided for amplifying all other polynucleotides, including all low abundance polynucleotides. In other embodiments primers are provided for amplifying at least 20, 30, 40, 50, 75, 100, 250, 500, 1000 or more low abundance polynucleotides and blocking oligomers are provided for blocking at least 20, 30, 40, 50, 75, 100, 250, 500, 1000 or more high abundance polynucleotides.

In other embodiments, the invention provides kits for labeling polynucleotide samples that have been enriched in low abundance species. These kits can provide the components listed above, and in addition, provide a means for labeling cRNA or cDNA molecules.

In still other embodiments, the present invention provides kits for the analysis of gene expression using the polynucleotide pools produced by the methods described herein. These kits can include components listed above, and in addition provide a labeling means and suitable hybridization probes affixed to a suitable array or chip, as well as reagents required for the detection/visualization of hybridized complexes.

In other embodiments, the invention provides cross hybridization assay kits, where the kits are useful for the analysis of probe specificity by determining the amount of probe cross hybridization exists in a sample that has been specifically depleted for the polynucleotide target sequence of interest. This information can be ascertained from samples from any source, including human samples.

In addition, kits of the present invention can also include, for example but not limited to, apparatus and reagents for sample collection and/or purification, apparatus and reagents for product collection and/or purification, sample tubes, holders, trays, racks, dishes, plates, instructions to the kit user, solutions, buffers or other chemical reagents, suitable samples to be used for standardization, normalization, and/or control samples. Kits of the present invention can also be packaged for convenient storage and shipping, for example, in a box having a lid.

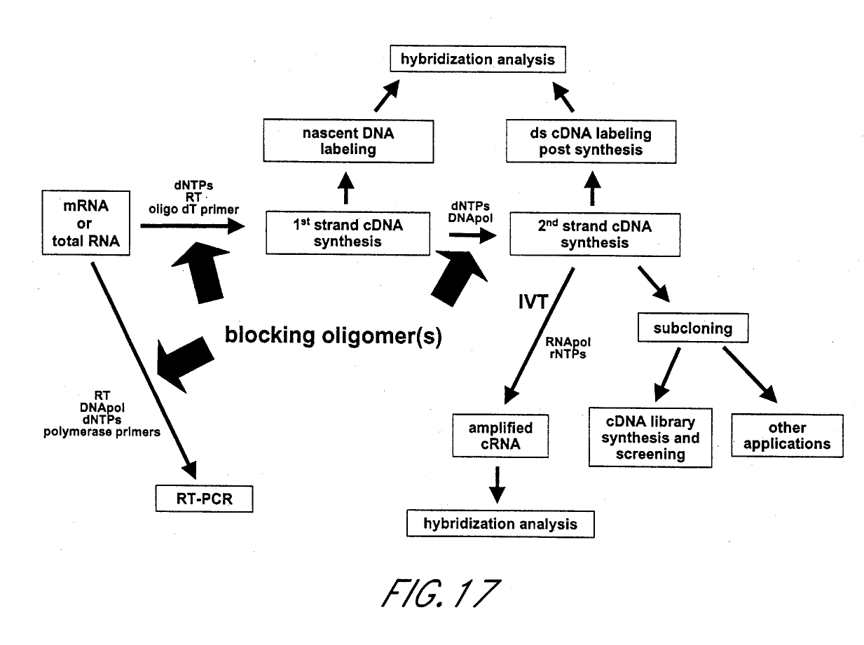

Some aspects of the invention are shown in FIG. 17. As shown in that figure, blocking oligomers can be utilized in various polymerase reactions, including but not limited to, reverse transcriptase reactions (e.g., cDNA first strand synthesis), second strand cDNA synthesis, and PCR reactions. Selected applications of the invention are also depicted in FIG. 17. These include, but are not limited to, hybridization/gene expression analysis, RT-PCR, cDNA library construction, cDNA library screening, and in vitro transcription. Other applications and uses for the invention not depicted in FIG. 17 are described elsewhere herein. Furthermore, it is intended that uses of the invention not specifically described herein, but would be recognized by one familiar with the art after reading the description of the invention, are also within the scope of the invention.

The following EXAMPLES are provided to further illustrate certain embodiments and aspects of the present invention. It is not intended that these EXAMPLES should limit the scope of any aspect of the invention. Although specific reaction conditions and reagents are described, it is clear that one familiar with the art would recognize alternative or equivalent conditions that also find use with the invention, where the alternative or equivalent conditions do not depart from the scope of the invention.

### EXAMPLE 1

### Reverse Transcription and First Strand cDNA Synthesis of an Artificial Gene Transcript in the Presence of Blocking PNA Oligomers

In this EXAMPLE, the ability of non-extendable PNA oligomers to block reverse transcriptase cDNA first strand synthesis was examined using an in vitro-generated artificial transcript corresponding to the ATP5F1 gene (GenBank Accession Number NM_{—}001688; human import precursor of subunit B of the H⁺ transporting, mitochondrial ATP synthase, isoform 1). Blocking oligomers specific for the ATP5F1 gene of various length and sequence were tested in this assay.

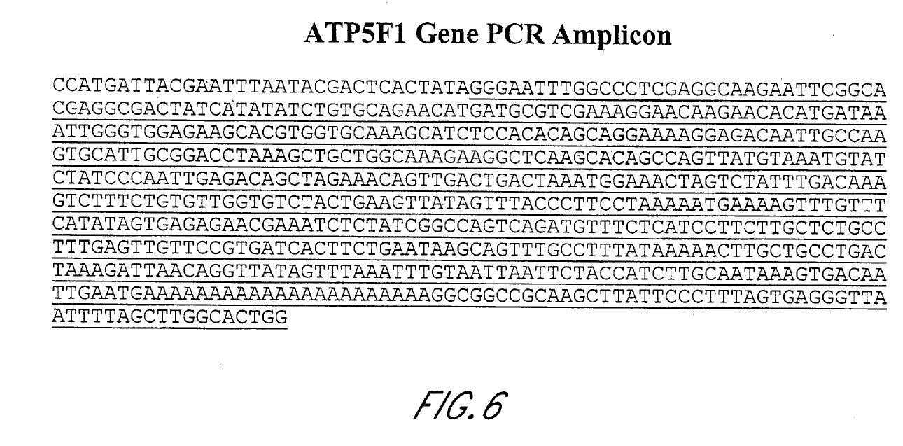

Artificial truncated transcripts of the ATP5F1 gene 636 ribonucleotides in length were generated by in vitro transcription using T7 RNA polymerase from a PCR amplicon as template. The complete sequence for the ATP5F1 PCR amplicon is provided in FIG. 6 and SEQ ID NO: 1. The portion of the ATP5F1 gene used as the artificial transcript was nucleotides 33-658, and are shown underlined in FIG. 6. Various PNA oligomers were designed and synthesized to be complementary to several different regions of the artificial ATP5F1 transcript, including overlapping the first 3 A's of the polyA tail, 3 bases upstream from the polyA tail, and other sites internal to the gene. PNA oligomers were synthesized using a commercial solid-phase synthesis service (Applied Biosystems, Foster City, Calif.), and dissolved in 1% 1-methyl-2-pyrrolidinone (N-methylpyrrolidone; NMP) in water to a concentration of 50 μM, as measured by Abs₂₆₀. The ATP5F1 PNA oligomers synthesized are shown in FIG. 8 and SEQ ID NOS: 3-20.

Reverse transcription reactions were run by first combining 2.0 μg ATP5F1 transcript template and 50 pmoles PNA oligomer in a final volume of 10.5 μL. The mixture was heated to 95° C. for 5 minutes, then cooled to 4° C. To this mix was added either 50 pmoles oligo-dT₂₁ deoxyribonucleotide RT primer or water to a final volume of 11.5 μL. This primer has the sequence:

5′-TTTTTTTTTTTTTTTTTTTTT-3′ (SEQ ID NO: 41)

The mixture was heated to 70° C. for 5 minutes, then cooled to 4° C. Using this annealed mix, the RT reactions were performed in a 20 μL reaction volume comprising 0.4 μM ATP5F1 RNA template, 2.5 μM PNA oligomer, 2.5 μM oligo-dT₂₁ primer, 1 mM each dATP, dCTP, dGTP, and dCTP, 10 mM DTT, 1×GibcoBRL® SUPERSCRIPT II™buffer, and 5 Units/μL GibcoBRL® SUPERSCRIPT II™ reverse transcriptase.

The reaction was carried out at 42° C. for 1 hour, followed by heat inactivation at 70° C. for 15 minutes. RNA template was hydrolyzed by the addition of 2 μL 2.5 M NaOH and incubation at 37° C. for 15 minutes. The reaction mix was neutralized by the addition of 20 μL 1 M Tris, pH 7.0. The single-stranded cDNA in the sample was purified with QIAGEN® QIAquick™ DNA purification spin column following the manufacturer's instructions.

One eighth of the purified DNA product from the RT reaction was resolved by agarose gel electrophoresis and detected using ethidium bromide staining, as shown in FIG. 11. Lane 12 shows the single-stranded ATP5F1 RNA template, approximately 600 ribonucleotides in length. Lane 10 shows the single-stranded deoxyribonucleotide product of reverse transcription in the absence of any PNA oligonucleotide, revealing a single predominant product approximately 600 nucleotides in length. The inhibitory effect of the various PNA oligomers can be clearly observed. All PNA oligomers tested, including others not shown on this ethidium gel, showed some ability to block cDNA first strand synthesis. PNA numbers 859 and 864 are the same length and have the same predicted Tₘ, however, 864, which binds the first 3 bases of the polyA tail, appears to have a slightly stronger blocking effect. Reactions with PNA numbers 869 and 873, which bind 235 and 345 nucleotides, respectively, from the polyA tail appear to produce small amounts of truncated single-stranded cDNA of approximately those sizes. Using more than one PNA blocker can increase the degree of RT product inhibition. Lanes 8 and 9 demonstrate that using two or three PNA sequences in concert in a single RT reaction further improves blocking efficiency, where no cDNA product was detectable in these reactions. Lane 11 contains 1-Kb ladder DNA size markers (Invitrogen™/Life Technologies™ Catalog No. 10787-018).

In order to demonstrate that this inhibitory effect was due to blocking of the reverse transcriptase by the PNA oligomers, control experiments using the ATP5F1 transcript template were performed, and the results shown in FIG. 12. First, it was tested whether the 1% NMP solvent used to dissolve the PNAs was able to inhibit the RT reaction. FIG. 12, lane 8 shows that a final concentration of 0.05% NMP in the RT reaction had no effect on RT activity and the generation of cDNA product.

It was also tested whether the RT inhibition observed was dependent on the dose of PNA oligomer. FIG. 12, lanes 2-7, show the effects of a range of PNA concentrations in the RT reaction products. PNA oligomer number 864 was used in two-fold serial dilutions. In each of these reactions, the molar concentration of the ATP5F1 transcript template was 0.4 μM. When the PNA oligonucleotide concentration is raised above 0.5 μM, inhibition is observed, suggesting a one-to-one stoichiometry of PNA binding to its target. In FIG. 12, lane 1 contains 1-Kb ladder DNA size markers (Invitrogen™/Life Technologies™ Catalog No. 10787-018), and lane 11 contains RNA ladder markers (Life Technologies™ Catalog No. 15620-016).

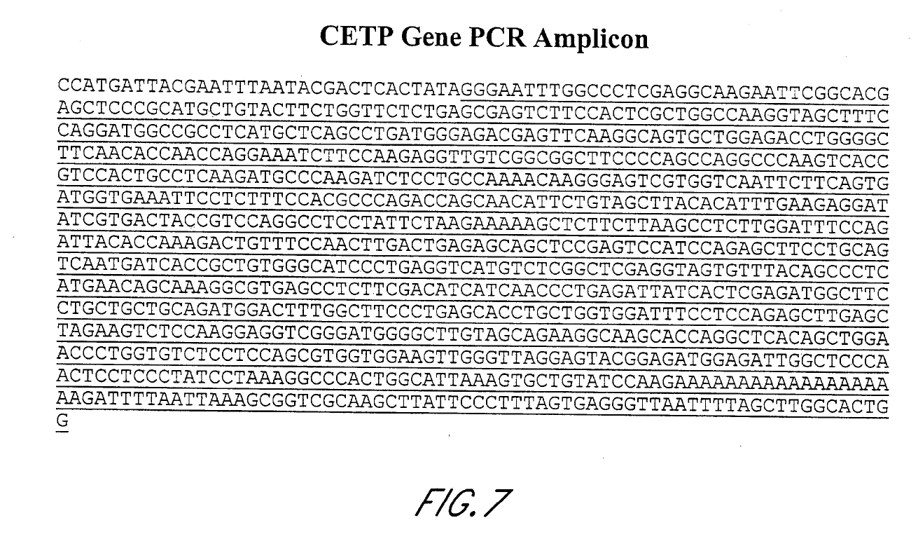

In order to demonstrate the sequence specificity of the blocking activity, the same ATP5F1 PNA oligonucleotide dilution series was used in RT reactions with a heterologous RNA template generated from the CETP gene. Artificial truncated transcripts of the CETP gene 959 ribonucleotides in length were generated by in vitro transcription using T7 RNA polymerase from a PCR amplicon as template. The complete sequence for the CETP amplicon is provided in FIG. 7 and SEQ ID NO: 2. The portion of the CETP amplicon used as the artificial transcript was nucleotides 33-991, and are shown underlined in FIG. 7.

The results of this experiment using a heterologous transcript are shown in FIG. 13. In each of these reactions the final concentration of CETP transcript template was 0.3 μM. Even at the highest concentration of ATP5F1-specific PNA oligonucleotide (2.5 μM), there is no inhibition of the CETP RT reaction, indicating that the blocking is sequence-specific and not due to non-specific interference. In FIG. 13, lane 1 contains 1-Kb ladder DNA size markers (Invitrogen™/Life Technologies™ Catalog No. 10787-018), and lane 11 contains RNA ladder markers (Life Technologies™ Catalog No. 15620-016).

### EXAMPLE 2

### Reverse Transcription and Double-Stranded cDNA Synthesis in the Presence of Blocking PNA Oligomers

This EXAMPLE describes the generation of double-stranded cDNAs from starting samples of total RNA and polyA RNA (i.e., mRNA), where the amplification of two target transcripts in the RNA sample was simultaneously blocked using blocking PNA oligomers.

In these RT reactions, a total of 0.05-1.0 μg mRNA or 2-10 μg total RNA isolated from human liver tissue (Ambion, Inc., Austin, Tex.; polyA RNA catalog number 7961, total RNA catalog number 7960) was used in a 20 μL reaction volume in a 1×RT reaction buffer (Applied Biosystems, High Capacity cDNA Archive Kit, Product No. 4322171). Each of the RT reactions contained 5 μM of a oligo-dT primer comprising sequence that hybridizes to the polyA sequence in the mRNA and also contains the T7 promoter consensus sequence. This primer, termed T7-dT₂₄, has the sequence:

| | |
|---|---|
| 5′-CGAATTTAATACGACTCACTATAGGGAGATTTTTTTTTTTTTTTTTTTTTTTT-3′ | (SEQ ID NO: 42) |

In addition, a separate set of reactions was also run, similar to the conditions above, but with the addition of four different PNA blocking oligomers, two of which are predicted to hybridize to the endogenous ATP5F1 transcript and two of which are predicted to hybridize to the endogenous CETP transcript. The ATP5F1-specific PNA oligomers used in this experiment were numbers 859 and 875 (see, FIG. 8, and SEQ ID NOS: 4 and 20), respectively. The CETP-specific PNA oligomers used in the experiment were numbers 849 and 854 (see, FIG. 9, and SEQ ID NOS: 31 and 36), respectively. Each of the PNA blockers were added to the RT reaction at a final concentration of 2.5 μM each.

The RT reaction mixtures were denatured at 70° C. for 5 min. First strand cDNA synthesis was performed by the addition of 100-200 U reverse transcriptase (recombinant MoMuLV MultiScribe™ Reverse Transcriptase, Applied Biosystems, Foster City, Calif.), 1 mM dNTPs and 30 U RNase inhibitor (Applied Biosystems, Catalog No. N808-0119) and incubated at 42° C. for 2 hours. The RT reaction was terminated by heating at 65° C. for 15 min. Excess RT primer was removed from the reaction using a MICROCON®-100 filtration column (Millipore Corporation, Bedford, Mass.).

Second strand cDNA was synthesized using a DNA-dependent DNA polymerase and random DNA primers. The reaction comprised 1000 μM each dNTP, 20 μM 5′-phosphorylated random 8-9 mers, 0.1-1 U/μL Bst DNA polymerase, and 16 U/μL T4 DNA ligase at 37° C. for 2 hours. The resulting double-stranded cDNA was made blunt-ended by treatment with 10-20 U of T4 DNA polymerase for 15 min at 37° C. Blunt-end, double-stranded cDNA was purified by filtration column (MICROCON®-100, Millipore Corporation) or affinity capture column (QIAGEN® QIAquik™ purification kit).

### EXAMPLE 3

### In Vitro Transcription and Generation of cRNA From cDNA

In this EXAMPLE, the double-stranded cDNA generated as described in EXAMPLE 2 is used in an in vitro transcription (IVT) reaction to generate cRNA products Two different reactions are described in this EXAMPLE. In one reaction, the IVT reaction produces unlabeled cRNA products, suitable for use in subsequent real-time PCR quantitation (i.e., TaqMan® analysis; see EXAMPLE 4). In the second reaction, labeled cRNA products are produced by incorporating a fluorescently labeled ribonucleotide into the nascent cRNA chain, producing a pool of labeled products suitable for use in high-throughput hybridization screening (i.e., array format probing; see EXAMPLE 5).

Both of the IVT reactions were run using the T7-promoter-containing double-stranded cDNA as a template and T7 RNA polymerase to initiates transcription from the T7 promoter sequence at the 3′ end of the cDNA. The reactions were conducted in 20-μL volumes, and contained 10-40 U/μL T7 RNA polymerase, 20 mM MgCl₂, 40 mM Tris-HCl, pH 8.0, 10 mM DTT and 2 mM spermidine. The IVT reaction used 7.5 mM each of ATP, CTP, GTP and UTP to produce unlabeled cRNA. A separate set of IVT reactions contained 7.5 mM each of ATP, CTP and GTP, and a reduced amount of UTP, and in addition, also contained 0.5-2.5 mM dye-linker UTP. The IVT reactions were allowed to proceed at 37° C. for 6-9 hours. The amplified cRNAs were purified using a QIAGEN® RNeasy® total RNA purification column to remove unincorporated ribonucleotides.

### EXAMPLE 4

### Real-Time Quantitative PCR Monitoring of cRNA Products

This EXAMPLE describes the quantitation of specific cRNA products in the unlabeled cRNA pool generated as described in EXAMPLE 3. This EXAMPLE utilized a TaqMan® RNA quantitation protocol, as commonly used in the art. The effectiveness of the PNA oligomers to block the amplification of various target transcripts in a sequence-specific manner in the reverse transcriptase step was assessed. The results of this analysis are shown in FIGS. 15-16.

The cRNA generated following RT-IVT amplification without the incorporation of fluorescent dye-linked UTP (see EXAMPLE 3) was used in a real-time PCR quantitation assay using a TaqMan protocol. The cRNA products from a total of four non-targeted genes, ATP5B, COX6B, RPS4X, PEX7, and the two targeted genes, ATP5F1 and CETP (as described in EXAMPLE 2), were quantitated. Quantitation was by RT-PCR using the cRNA as template, coupled with TaqMan® analysis.

PCR primers and double dye-labeled TaqMan® probes were designed using Primer Express™ (Version 1.0, Applied Biosystems, Foster City, Calif.). The Tₘ of the PCR primers ranged from 58° C. to 60° C., and the Tₘ of the TaqMan® probes ranged from 68° C. to 70° C.

PCR amplification reactions (50 μL) contained 10,000×diluted cRNA sample generated by IVT as described in EXAMPLE 3, 2×master mix (25 μL), which included PCR buffer, dNTPs, and MgCl₂, MuLV reverse transcriptase, AmpliTaq Gold® DNA polymerase (Applied Biosystems, Foster City, Calif.), gene-specific forward and reverse primers (200 to 900 nM each), and a TaqMan® probe (200-250 nM). The PCR primers and TaqMan® probe sequences used in these reactions are shown in TABLE 2.

TABLE 2

SEQ ID NO

ATB5B

| | | |
|---|---|---|
| forward PCR primer | 5′-GCTGAGACAAGAAACGCTGTATTTT-3′ | 43 |
| ATP5B-87F | | |
| reverse PCR primer | 5′-TGGATGAACTTTCTGAGGAAGACA-3′ | 44 |
| ATP5B-87R | | |
| TaqMan ® probe | FAM-CGTGCACGGGACACGGTCAACT-TMR | 45 |
| IcTaqMan | | |

COX6B

| | | |
|---|---|---|
| forward PCR primer | 5′-GAAGCGGCTGTCAAAAGGG-3′ | 46 |
| COX6B-403Tqman-R59 | | |
| reverse PCR primer | 5′-CTGCAGGTTGAATCCGGG-3′ | 47 |
| COX6B399-F59 | | |
| TaqMan ® probe | 6FAM-TGATTTTGGTCTCCATGTCTTCCGCC-TAMRA | 48 |
| 6bR-TaqMan | | |

RPS4X

| | | |
|---|---|---|
| forward PCR primer | 5′-ATTTTTAATTACGTACAAAGATCTGACATGT-3′ | 49 |
| RPS4X-94F | | |
| reverse PCR primer | 5′-AGAGACAAAAGACTGGCGGC-3′ | 50 |
| RPS4X-94R | | |
| TaqMan ® probe | FAM-CCATTTCACCCACTGCTCTGTTTGG-TMR | 51 |
| 17aTaqMan | | |

PEX7

| | | |
|---|---|---|
| forward PCR primer | 5′-TGAGTTGTGACTGGTGTAAATACAATGA-3′ | 52 |
| pex7-F | | |
| reverse PCR primer | 5′-AAGTCCCAGCCTCTCAAACTACAG-3′ | 53 |
| pex7-R | | |
| TaqMan ® probe | 6FAM-CCCGGTCACCAGCAA-MGB | 54 |
| pex7-probe | | |

ATP5F1

| | | |
|---|---|---|
| forward PCR primer | 5′-TGAGCCTTCTTTGCCAGCA-3′ | 55 |
| ATP5F1-89F | | |
| reverse PCR primer | 5′-CACAGCAGGAAAAGGAGACAATT-3′ | 56 |
| ATP5F1-89R | | |
| TaqMan ® probe | FAM-AAGGATGAGAAACATCTGACTGGCCGATAGA-TMR | 57 |
| 2aTaqMan | | |

CETP

| | | |
|---|---|---|
| forward PCR primer | 5′-GCTCACGCCTTTGCTGTTC-3′ | 58 |
| CETP-90F | | |
| reverse PCR primer | 5′-TCACCGCTGTGGGCATC-3′ | 59 |
| CETP-90R | | |
| TaqMan ® probe | FAM-TAAACACTACCTCGAGCCGAGACATGACCT-TMR | 60 |
| 5aTaqMan | | |

The RT-PCR reaction conditions included 45 min at 50° C. and then 10 min at 95° C. RT-PCR thermal cycling proceeded with 40 cycles of 95° C. for 15 sec and 60° C. for 1 min. All reactions were performed in an ABI PRISM® 7700 Sequence Detection System (Applied Biosystems, Foster City, Calif.). Software for data collection and analysis were Applied Biosystems products.

The results of this TaqMan® analysis are shown in FIG. 15. Results are expressed as C_{T}, or the threshold cycle. Fluorescence values are recorded during every cycle and represent the amount of product amplified to that point in the amplification reaction. The point when the fluorescent signal is first recorded as statistically significant is the threshold cycle (C_{T}). Following analysis and C_{T} calibration against stardardization values (data not shown), it was determined that these data demonstrate that PNA oligomers can effectively block the transcription of specific target genes (ATP5F1 and CEPT) by 99.1 and 99.6% during RT and IVT amplification using either mRNA or total cellular RNA starting material as template, respectively. Furthermore, these data also demonstrate that these same blocking PNA oligomers used to inhibit the ATP5F1 and CEPT reverse transcriptase reactions do not inhibit the reverse transcription of the non-targeted genes (i.e., ATP5B, COX6B, RPS4X and PEX7). The data shown in FIG. 15 is also shown graphically in FIG. 16.

### EXAMPLE 5

### Enrichment of Low Abundance Transcripts in a Sample Using 2′-O-methyl Ribonucleotide Blocking Oligomers

This EXAMPLE describes the generation of double-stranded cDNAs from a starting sample of human liver polyA RNA (i.e., mRNA), where the resulting cDNA pool is enriched in low abundance transcripts by blocking the amplification of the high abundance 13-actin transcript using specific 2′-O-methyl ribonucleotide blocking oligomers.

In this method, a total of 1.0 μg polyA mRNA isolated from human liver tissue (Ambion, Inc., Austin, Tex.; catalog number 7961) is used in a 20 μL reverse transcriptase reaction. This RT reaction uses a 1×RT reaction buffer (Applied Biosystems, High Capacity cDNA Archive Kit, Product No. 4322171), and 5 μM of an oligo-dT primer, termed T7-dT₂₄, (SEQ ID NO: 42).

In addition, the reaction also contains at least one 2′-O-methyl ribonucleotide blocking oligomer comprising a nucleobase sequence that is capable of hybridizing to the β-actin mRNA transcript (GenBank Accession Number NM_{—}001101). The 2′-O-methyl ribonucleotide oligomers are synthesized using standard phosphoramidite chemistry using 2′-O-methylphosphoramidites (A, G, C and U), which are available from various commercial sources (e.g., Glen Research Corporation, Sterling, Va.), and are purified using standard polyacrylamide gel electrophoresis.

Examples of β-actin-specific 2′-O-methyl ribonucleotide blocking oligomers include, but are not limited to:

| | |
|---|---|
| 5′-AUGCUAUCACCUCCCCUGUG-3′ | (SEQ ID NO: 61) |
| 5′-UCAAGUUGGGGGACAAAAAG-3′ | (SEQ ID NO: 62) |
| 5′-AGUGGGGUGGCUUUUAGGAU-3′ | (SEQ ID NO: 63) |
| 5′-UUUUUAAGGUGUGCACUUUU-3′ | (SEQ ID NO: 64) |

Any one of these blocking oligomers can be used in the RT reaction, or alternatively, any combination of the oligomers can be used, including all of the oligomers simultaneously in the same reaction. Each of the 2′-O-methyl ribonucleotide blocking oligomers is added to the RT reaction to a final concentration of 2.5 μM each.

The RT reaction mixture is denatured at 70° C. for 5 min. First strand cDNA synthesis is performed by the addition of 100-200 U reverse transcriptase (e.g., recombinant MoMuLV MultiScribe™ Reverse Transcriptase, Applied Biosystems, Foster City, Calif.), 1 mM dNTPs and 30 U RNase inhibitor (e.g., Applied Biosystems, Catalog No. N808-0119) and incubated at 42° C. for 2 hours. The RT reaction is terminated by heating at 65° C. for 15 min. Excess RT primer is removed from the reaction using a MICROCON®-100 filtration column (Millipore Corporation, Bedford, Mass.).

Second strand cDNA is synthesized using a DNA-dependent DNA polymerase and random DNA primers. This reaction comprises 1000 μM each dNTP, 20 μM 5′-phosphorylated random 8-9 mers, 0.1-1 U/μL Bst DNA polymerase, and 16 U/μL T4 DNA ligase at 37° C. for 2 hours. The resulting double-stranded cDNA is made blunt-ended by treatment with 10-20 U of T4 DNA polymerase for 15 min at 37° C. Blunt-end, double-stranded cDNA is purified by filtration column (MICROCON®-100, Millipore Corporation) or affinity capture column (QIAGEN® QIAquik™ purification kit).

All publications, GenBank Accession Number sequence submissions, patents and published patent applications mentioned in the above specification are herein incorporated by reference in their entirety. Various modifications and variations of the described compositions and methods of the invention will be apparent to those skilled in the art without departing from the scope and spirit of the invention. Although the invention has been described in connection with various specific embodiments, it should be understood that the invention as claimed should not be unduly limited to such specific embodiments. Indeed, various modifications of the described modes for carrying out the invention which are obvious to those skilled in gene expression analysis and nucleic acid enzymology and biochemistry or related fields are intended to be within the scope of the following claims.

What is claimed is:

1. A method for the enrichment of a low abundance polynucleotide relative to a high abundance polynucleotide in a sample, wherein the ratio of the high abundance polynucleotide to the low abundance polynucleotide is at least about 10:1, the method comprising
(a) exposing said sample to at least one first enzymatically non-extendable nucleobase oligomer having a nucleobase sequence complementary to a sequence within the high abundance polynucleotide under conditions such that base pairing occurs;
(b) exposing said sample to a primer having a nucleobase sequence complementary to a sequence within the low abundance polynucleotide under conditions such that base pairing occurs; and
(c) subjecting said sample to conditions for polymerase extension, such that said low abundance polynucleotide is amplified by extension of the primer and the high abundance polynucleotide is not amplified.

2. The method of claim 1, wherein the ratio of the high abundance polynucleotide to the low abundance polynucleotide is at least 100:1.

3. The method of claim 1, wherein the sample comprises at a first and a second high abundance polynucleotide and in step (a) is exposed to at least two enzymatically non-extendable nucleobase oligomers, wherein one nucleobase oligomer comprises a nucleobase sequence that is complementary to a sequence within the first high abundance polynucleotides and the second nucleobase oligomer comprises a nucleobase sequence that is complementary to a sequence within the second high abundance polynucleotide.

4. The method of claim 1, wherein the low abundance polynucleotide and high abundance polynucleotide are RNA molecules selected from the group consisting of mRNA, rRNA, cRNA and tRNA molecules.

5. The method of claim 1, wherein the low abundance and high abundance polynucleotides are cDNA molecules.

6. The method of claim 1, wherein said enzymatically non-extendable nucleobase oligomer does not have a ribose-containing oligomeric structure.

7. The method of claim 6, wherein said enzymatically non-extendable nucleobase oligomer is a peptide nucleic acid (PNA) oligomer.

8. The method of claim 1, wherein said enzymatically non-extendabale nucleobase oligomer is a modified nucleotide oligomer or internucleotide analog oligomer.

9. The method of claim 8, wherein said modified nucleotide oligomer is selected from the group consisting of 2′-modified and 3′-modified nucleotide oligomers.

10. The method of claim 9, wherein said 2′-modified and 3′-modified nucleotide oligomer is selected from the group consisting of 2′-O-alkyl modified nucleotide oligomers and 3′-alkyl modified nucleotide oligomers.

11. The method of claim 10, wherein said 2′-O-alkyl modified nucleotide oligomers are 2′-o-methyl nucleotide oligomers.

12. The method of claim 8, wherein said modified nucleotide oligomer or internucleotide analog oligomer is selected from locked nucleic acids (LNA), N3′-P5′ phosphoramidate (NP) oligomers, minor groove binder-linked-oligonucleotides (MGB-linked oligonucleotides), phosphorothioate (PS) oligomers, C₁-C₄ alkylphosphonate oligomers, phosphoramidates, β-phosphodiester oligonucleotides, and a-phosphodiester oligonucleotides.

13. The method of claim 12, wherein said C₁-C₄ alkylphosphonate oligomers are methyl phosphonate (MP) oligomers.

14. The method of claim 1, wherein said enzymatically non-extendable first nucleobase oligomer is chimeric.

15. The method of claim 1, wherein said sample comprises more than one high abundance polynucleotide.

16. The method of claim 1, wherein said sample of polynucleotides comprises polynucleotides selected from the group consisting of RNA and DNA.

17. The method of claim 1, wherein said sample of polynucleotides comprises RNA, and polymerase extension is by reverse transcription to yield a first strand cDNA.

18. The method of claim 17, wherein said method further comprises second strand cDNA synthesis.

19. The method of claim 18, wherein said sample is exposed to at least one enzymatically non-extendable nucleobase oligomer during first strand cDNA synthesis.

20. The method of claim 18, wherein said sample is exposed to at least one enzymatically non-extendable nucleobase oligomer during second strand cDNA synthesis.

21. The method of claim 18, wherein said sample is exposed to at least one enzymatically non-extendable nucleobase oligomer during both first strand cDNA synthesis and second strand cDNA synthesis.

22. The method of claim 18, wherein said method further comprises an amplification step.

23. The method of claim 22 wherein said amplification step is by polymerase chain reaction.

24. The method of claim 22 wherein said amplification step is by in vitro transcription.

25. The method of claim 16 wherein said RNA is mRNA or cRNA or total cellular RNA.

26. The method of claim 1 wherein said sample of polynucleotides comprises DNA, and polymerase extension is by DNA-dependent DNA-polymerase in a polymerase chain reaction.

27. The method of claim 22, further comprising a step of labeling said amplified polynucleotides.

28. The method of claim 27, wherein said labeling is concomitant with amplification.

29. The method of claim 27, wherein said labeling is subsequent to amplification.

30. A plurality of polynucleotides, where the relative abundance of at least one target polynucleotide has been reduced relative to a non-target polynucleotide, and wherein at least one target polynucleotide is selected from the list of genes recited in FIG. 14.

31. The plurality of polynucleotides of claim 30, where the relative abundance of at least one non-target polynucleotide has been increased relative to a target polynucleotide.

32. The plurality of polynucleotides of claim 30, where the plurality of polynucleotides are DNA molecules or RNA molecules.

33. The plurality of polynucleotides of claim 32, where the DNA molecules are cDNA molecules.

34. The plurality of polynucleotides of claim 32, where the RNA molecules are cRNA molecules.

35. The plurality of polynucleotides of claim 30, where the polynucleotides are labeled.

36. The plurality of polynucleotides of claim 33, where the cDNA molecules are cloned into a vector.

37. A kit for the enrichment of at least one low abundance polynucleotide in a sample of polynucleotides, wherein said sample comprises at least one high abundance polynucleotide and at least one low abundance polynucleotide, wherein said kit comprises at least one enzymatically non-extendable nucleobase oligomer having a nucleobase sequence complementary to said at least one high abundance target polynucleotide.

38. The kit of claim 37, wherein the sample comprises at least 5 high abundance polynucleotides and the kit comprises at least five non-enzymatically non-extendable nucleobase oligomers each having a nucleobase sequence complementary to one of the five high abundance target polynucleotides.

39. The kit of claim 37, additionally comprising a primer for amplifying the at least one low abundance polynucleotide.

40. The method of claim 39, wherein the primer is a random primer.

41. The kit of claim 37, wherein said high abundance target polynucleotide is selected from the genes recited in FIG. 14.

42. The kit of claim 37, wherein said non-extendable nucleobase oligomer is selected from peptide nucleic acid (PNA) oligomers, 2′-O-alkyl modified nucleotide oligomers, 3′-alkyl modified nucleotide oligomers, locked nucleic acids (LNA), N3′-P5′ phosphoramidate (NP) oligomers, minor groove binder-linked-oligonucleotides (MGB-linked oligonucleotides), phosphorothioate (PS) oligomers, C₁-C₄ alkylphosphonate oligomers, phosphoramidates, 13-phosphodiester oligonucleotides, and a-phosphodiester oligonucleotides.

43. The kit of claim 37, further comprising one or more components selected from the group consisting of an RNA-dependent DNA polymerase (reverse transcriptase), a DNA-dependent RNA polymerase, a DNA-dependent DNA polymerase, an oligo-dT polymerase primer, an oligo-dT polymerase primer further comprising nucleotide sequence for RNA polymerase initiation, deoxyribonucleotide triphosphates, ribonucleotide triphosphates, a DNA polymerase primer suitable for cDNA second strand synthesis, and a means for polynucleotide labeling.

44. A method for analyzing gene expression in a sample having at least one high abundance polynucleotide, comprising:
(a) exposing said sample to at least one enzymatically non-extendable nucleobase oligomer having a nucleobase sequence complementary to a sequence within said high abundance polynucleotide under conditions such that base pairing occurs,
(b) subjecting said sample to conditions for polymerase extension to produce an enriched polynucleotide sample,
(c) labeling said polynucleotides in said enriched polynucleotide sample,
(d) contacting said labeled polynucleotide sample with a probe using a hybridization means to form a hybridization complex, and
(e) detecting said hybridization complex, where the detection of a hybridization complex is indicative of gene expression.

45. A method for the synthesis of a cDNA library enriched for at least one low abundance polynucleotide, comprising the steps of:
(a) providing a sample of mRNA, where said mRNA has at least one high abundance transcript and at least one low abundance transcript,
(b) exposing said sample to at least one enzymatically non-extendable nucleobase oligomer having a nucleobase sequence complementary to a sequence within said high abundance mRNA under conditions such that base pairing occurs,
(c) subjecting said sample to conditions for reverse transcription and first strand cDNA synthesis,
(d) subjecting said sample to conditions for second strand cDNA synthesis to form double stranded cDNA molecules,
(e) cloning said double stranded cDNA molecules into a vector to yield an enriched cDNA library.

46. A method of enriching a sample for one or more low abundance polynucleotides comprising:
amplifying the low abundance polynucleotides using polymerase extension while blocking amplification of at least one high abundance polynucleotide,
wherein blocking amplification of the high abundance polynucleotide comprises contacting the high abundance polynucleotide prior to amplification with an enzymatically non-extendable oligomer comprising a sequence that is complementary to a sequence within the high abundance polynucleotide under conditions such that base pairing occurs, and
wherein the ratio of the high abundance polynucleotide to each low abundance polynucleotide is at least about 10:1.

47. The method of claim 46 wherein the sample is enriched for at least 10 low abundance polynucleotides.

48. The method of claim 46 wherein the sample is enriched for at least 100 low abundance nucleotides.

49. The method of claim 46 wherein amplification of at least 2 high abundance polypeptides is blocked.

50. The method of claim 46 wherein amplification of at least 10 high abundance polypeptides is blocked.

51. The method of claim 46 wherein amplification of at least 50 high abundance polypeptides is blocked.

52. The method of claim 46 wherein the sample is enriched for at least 10 low abundance polynucleotides and the amplification of at least 2 high abundance polypeptides is blocked.


SEQUENCE LISTING (64 sequences)

SEQ ID NO: 1 - DNA, 658 bp, Homo sapiens
  gene (1)...(658): Sequence derived from the human ATP5F1 gene.
ccatgattacgaatttaatacgactcactatagggaatttggccctcgaggcaagaattcggcacgaggcgactatcatatatctgtgcagaacatgatgcgtcgaaaggaacaagaacacatgataaattgggtggagaagcacgtggtgcaaagcatctccacacagcaggaaaaggagacaattgccaagtgcattgcggacctaaagctgctggcaaagaaggctcaagcacagccagttatgtaaatgtatctatcccaattgagacagctagaaacagttgactgactaaatggaaactagtctatttgacaaagtctttctgtgttggtgtctactgaagttatagtttacccttcctaaaaatgaaaagtttgtttcatatagtgagagaacgaaatctctatcggccagtcagatgtttctcatccttcttgctctgcctttgagttgttccgtgatcacttctgaataagcagtttgcctttataaaaacttgctgcctgactaaagattaacaggttatagtttaaatttgtaattaattctaccatcttgcaataaagtgacaattgaatgaaaaaaaaaaaaaaaaaaaaaaggcggccgcaagcttattccctttagtgagggttaattttagcttggcactgg

SEQ ID NO: 2 - DNA, 991 bp, Homo sapiens
  gene (1)...(991): Sequence derived from the human CETP gene.
ccatgattacgaatttaatacgactcactatagggaatttggccctcgaggcaagaattcggcacgagctcccgcatgctgtacttctggttctctgagcgagtcttccactcgctggccaaggtagctttccaggatggccgcctcatgctcagcctgatgggagacgagttcaaggcagtgctggagacctggggcttcaacaccaaccaggaaatcttccaagaggttgtcggcggcttccccagccaggcccaagtcaccgtccactgcctcaagatgcccaagatctcctgccaaaacaagggagtcgtggtcaattcttcagtgatggtgaaattcctctttccacgcccagaccagcaacattctgtagcttacacatttgaagaggatatcgtgactaccgtccaggcctcctattctaagaaaaagctcttcttaagcctcttggatttccagattacaccaaagactgtttccaacttgactgagagcagctccgagtccatccagagcttcctgcagtcaatgatcaccgctgtgggcatccctgaggtcatgtctcggctcgaggtagtgtttacagccctcatgaacagcaaaggcgtgagcctcttcgacatcatcaaccctgagattatcactcgagatggcttcctgctgctgcagatggactttggcttccctgagcacctgctggtggatttcctccagagcttgagctagaagtctccaaggaggtcgggatggggcttgtagcagaaggcaagcaccaggctcacagctggaaccctggtgtctcctccagcgtggtggaagttgggttaggagtacggagatggagattggctcccaactcctccctatcctaaaggcccactggcattaaagtgctgtatccaagaaaaaaaaaaaaaaaaaaagattttaattaaagcggtcgcaagcttattccctttagtgagggttaattttagcttggcactgg

SEQ ID NO: 3 - DNA, 23 bp, Artificial Sequence
  Peptide Nucleic Acid (PNA) using a 2-aminoethylglycine backbone, where the nucleobase sequence is derived from human sequence.
nntcaattgtcactttattgcan

SEQ ID NO: 4 - DNA, 21 bp, Artificial Sequence
  Peptide Nucleic Acid (PNA) using a 2-aminoethylglycine backbone, where the nucleobase sequence is derived from human sequence.
nntcaattgtcactttattgn

SEQ ID NO: 5 - DNA, 18 bp, Artificial Sequence
  Peptide Nucleic Acid (PNA) using a 2-aminoethylglycine backbone, where the nucleobase sequence is derived from human sequence.
ntcaattgtcactttatn

SEQ ID NO: 6 - DNA, 16 bp, Artificial Sequence
  Peptide Nucleic Acid (PNA) using a 2-aminoethylglycine backbone, where the nucleobase sequence is derived from human sequence.
ntcaattgtcactttn

SEQ ID NO: 7 - DNA, 14 bp, Artificial Sequence
  Peptide Nucleic Acid (PNA) using a 2-aminoethylglycine backbone, where the nucleobase sequence is derived from human sequence.
ntcaattgtcactn

SEQ ID NO: 8 - DNA, 23 bp, Artificial Sequence
  Peptide Nucleic Acid (PNA) using a 2-aminoethylglycine backbone, where the nucleobase sequence is derived from human sequence.
nntttcattcaattgtcactttn

SEQ ID NO: 9 - DNA, 20 bp, Artificial Sequence
  Peptide Nucleic Acid (PNA) using a 2-aminoethylglycine backbone, where the nucleobase sequence is derived from human sequence.
ntttcattcaattgtcactn

SEQ ID NO: 10 - DNA, 18 bp, Artificial Sequence
  Peptide Nucleic Acid (PNA) using a 2-aminoethylglycine backbone, where the nucleobase sequence is derived from human sequence.
ntttcattcaattgtcan

SEQ ID NO: 11 - DNA, 16 bp, Artificial Sequence
  Peptide Nucleic Acid (PNA) using a 2-aminoethylglycine backbone, where the nucleobase sequence is derived from human sequence.
ntttcattcaattgtn

SEQ ID NO: 12 - DNA, 14 bp, Artificial Sequence
  Peptide Nucleic Acid (PNA) using a 2-aminoethylglycine backbone, where the nucleobase sequence is derived from human sequence.
ntttcattcaattn

SEQ ID NO: 13 - DNA, 23 bp, Artificial Sequence
  Peptide Nucleic Acid (PNA) using a 2-aminoethylglycine backbone, where the nucleobase sequence is derived from human sequence.
nnacttcagtagacaccaacacn

SEQ ID NO: 14 - DNA, 20 bp, Artificial Sequence
  Peptide Nucleic Acid (PNA) using a 2-aminoethylglycine backbone, where the nucleobase sequence is derived from human sequence.
nacttcagtagacaccaacn

SEQ ID NO: 15 - DNA, 18 bp, Artificial Sequence
  Peptide Nucleic Acid (PNA) using a 2-aminoethylglycine backbone, where the nucleobase sequence is derived from human sequence.
nacttcagtagacaccan

SEQ ID NO: 16 - DNA, 16 bp, Artificial Sequence
  Peptide Nucleic Acid (PNA) using a 2-aminoethylglycine backbone, where the nucleobase sequence is derived from human sequence.
nacttcagtagacacn

SEQ ID NO: 17 - DNA, 14 bp, Artificial Sequence
  Peptide Nucleic Acid (PNA) using a 2-aminoethylglycine backbone, where the nucleobase sequence is derived from human sequence.
nacttcagtagacn

SEQ ID NO: 18 - DNA, 18 bp, Artificial Sequence
  Peptide Nucleic Acid (PNA) using a 2-aminoethylglycine backbone, where the nucleobase sequence is derived from human sequence.
nctgtgcttgagccttcn

SEQ ID NO: 19 - DNA, 18 bp, Artificial Sequence
  Peptide Nucleic Acid (PNA) using a 2-aminoethylglycine backbone, where the nucleobase sequence is derived from human sequence.
ntctttagtcaggcagcn

SEQ ID NO: 20 - DNA, 18 bp, Artificial Sequence
  Peptide Nucleic Acid (PNA) using a 2-aminoethylglycine backbone, where the nucleobase sequence is derived from human sequence.
nagatggtagaattaatn

SEQ ID NO: 21 - DNA, 23 bp, Artificial Sequence
  Peptide Nucleic Acid (PNA) using a 2-aminoethylglycine backbone, where the nucleobase sequence is derived from human sequence.
nntggatacagcactttaatgcn

SEQ ID NO: 22 - DNA, 20 bp, Artificial Sequence
  Peptide Nucleic Acid (PNA) using a 2-aminoethylglycine backbone, where the nucleobase sequence is derived from human sequence.
ntggatacagcactttaatn

SEQ ID NO: 23 - DNA, 18 bp, Artificial Sequence
  Peptide Nucleic Acid (PNA) using a 2-aminoethylglycine backbone, where the nucleobase sequence is derived from human sequence.
ntggatacagcactttan

SEQ ID NO: 24 - DNA, 16 bp, Artificial Sequence
  Peptide Nucleic Acid (PNA) using a 2-aminoethylglycine backbone, where the nucleobase sequence is derived from human sequence.
ntggatacagcacttn

SEQ ID NO: 25 - DNA, 14 bp, Artificial Sequence
  Peptide Nucleic Acid (PNA) using a 2-aminoethylglycine backbone, where the nucleobase sequence is derived from human sequence.
ntggatacagcacn

SEQ ID NO: 26 - DNA, 23 bp, Artificial Sequence
  Peptide Nucleic Acid (PNA) using a 2-aminoethylglycine backbone, where the nucleobase sequence is derived from human sequence.
nntttcttggatacagcactttn

SEQ ID NO: 27 - DNA, 20 bp, Artificial Sequence
  Peptide Nucleic Acid (PNA) using a 2-aminoethylglycine backbone, where the nucleobase sequence is derived from human sequence.
ntttcttggatacagcactn

SEQ ID NO: 28 - DNA, 18 bp, Artificial Sequence
  Peptide Nucleic Acid (PNA) using a 2-aminoethylglycine backbone, where the nucleobase sequence is derived from human sequence.
ntttcttggatacagcan

SEQ ID NO: 29 - DNA, 16 bp, Artificial Sequence
  Peptide Nucleic Acid (PNA) using a 2-aminoethylglycine backbone, where the nucleobase sequence is derived from human sequence.
ntttcttggatacagn

SEQ ID NO: 30 - DNA, 14 bp, Artificial Sequence
  Peptide Nucleic Acid (PNA) using a 2-aminoethylglycine backbone, where the nucleobase sequence is derived from human sequence.
ntttcttggatacn

SEQ ID NO: 31 - DNA, 23 bp, Artificial Sequence
  Peptide Nucleic Acid (PNA) using a 2-aminoethylglycine backbone, where the nucleobase sequence is derived from human sequence.
nncctcgagccgagacatgaccn

SEQ ID NO: 32 - DNA, 20 bp, Artificial Sequence
  Peptide Nucleic Acid (PNA) using a 2-aminoethylglycine backbone, where the nucleobase sequence is derived from human sequence.
ncctcgagccgagacatgan

SEQ ID NO: 33 - DNA, 18 bp, Artificial Sequence
  Peptide Nucleic Acid (PNA) using a 2-aminoethylglycine backbone, where the nucleobase sequence is derived from human sequence.
ncctcgagccgagacatn

SEQ ID NO: 34 - DNA, 16 bp, Artificial Sequence
  Peptide Nucleic Acid (PNA) using a 2-aminoethylglycine backbone, where the nucleobase sequence is derived from human sequence.
ncctcgagccgagacn

SEQ ID NO: 35 - DNA, 14 bp, Artificial Sequence
  Peptide Nucleic Acid (PNA) using a 2-aminoethylglycine backbone, where the nucleobase sequence is derived from human sequence.
ncctcgagccgagn

SEQ ID NO: 36 - DNA, 20 bp, Artificial Sequence
  Peptide Nucleic Acid (PNA) using a 2-aminoethylglycine backbone, where the nucleobase sequence is derived from human sequence.
ncacagcggtgatcattgan

SEQ ID NO: 37 - DNA, 18 bp, Artificial Sequence
  Peptide Nucleic Acid (PNA) using a 2-aminoethylglycine backbone, where the nucleobase sequence is derived from human sequence.
ncagcggtgatcattgan

SEQ ID NO: 38 - DNA, 18 bp, Artificial Sequence
  Peptide Nucleic Acid (PNA) using a 2-aminoethylglycine backbone, where the nucleobase sequence is derived from human sequence.
ntgaccacgactcccttn

SEQ ID NO: 39 - DNA, 18 bp, Artificial Sequence
  Peptide Nucleic Acid (PNA) using a 2-aminoethylglycine backbone, where the nucleobase sequence is derived from human sequence.
ncagcaggaagccatctn

SEQ ID NO: 40 - DNA, 17 bp, T7 bacteriophage
  promoter (0)...(0): bacteriophage T7 RNA polymerase promoter sequence
aatacgactcactatag

SEQ ID NO: 41 - DNA, 21 bp, Artificial Sequence
  oligo-dT primer
ttttttttttttttttttttt

SEQ ID NO: 42 - DNA, 53 bp, Artificial Sequence
  T7 promoter-oligo-dT primer
cgaatttaatacgactcactatagggagatttttttttttttttttttttttt

SEQ ID NO: 43 - DNA, 25 bp, Homo sapiens
gctgagacaagaaacgctgtatttt

SEQ ID NO: 44 - DNA, 24 bp, Homo sapiens
tggatgaactttctgaggaagaca

SEQ ID NO: 45 - DNA, 22 bp, Homo sapiens
  misc_feature (1)...(22): TaqMan probe
cgtgcacgggacacggtcaact

SEQ ID NO: 46 - DNA, 19 bp, Homo sapiens
gaagcggctgtcaaaaggg

SEQ ID NO: 47 - DNA, 18 bp, Homo sapiens
ctgcaggttgaatccggg

SEQ ID NO: 48 - DNA, 26 bp, Homo sapiens
  misc_feature (1)...(26): TaqMan probe
tgattttggtctccatgtcttccgcc

SEQ ID NO: 49 - DNA, 31 bp, Homo sapiens
atttttaattacgtacaaagatctgacatgt

SEQ ID NO: 50 - DNA, 20 bp, Homo sapiens
agagacaaaagactggcggc

SEQ ID NO: 51 - DNA, 25 bp, Homo sapiens
  misc_feature (1)...(25): TaqMan probe
ccatttcacccactgctctgtttgg

SEQ ID NO: 52 - DNA, 28 bp, Homo sapiens
tgagttgtgactggtgtaaatacaatga

SEQ ID NO: 53 - DNA, 24 bp, Homo sapiens
aagtcccagcctctcaaactacag

SEQ ID NO: 54 - DNA, 15 bp, Homo sapiens
  misc_feature (1)...(15): TaqMan probe
cccggtcaccagcaa

SEQ ID NO: 55 - DNA, 19 bp, Homo sapiens
tgagccttctttgccagca

SEQ ID NO: 56 - DNA, 23 bp, Homo sapiens
cacagcaggaaaaggagacaatt

SEQ ID NO: 57 - DNA, 31 bp, Homo sapiens
  misc_feature (1)...(31): TaqMan probe
aaggatgagaaacatctgactggccgataga

SEQ ID NO: 58 - DNA, 19 bp, Homo sapiens
gctcacgcctttgctgttc

SEQ ID NO: 59 - DNA, 17 bp, Homo sapiens
tcaccgctgtgggcatc

SEQ ID NO: 60 - DNA, 30 bp, Homo sapiens
  misc_feature (1)...(30): TaqMan probe
taaacactacctcgagccgagacatgacct

SEQ ID NO: 61 - RNA, 20 nt, Homo sapiens
  misc_feature (1)...(20): 2′-O-methyl ribonucleotide oligomer sequence, where A, U, G and C are modified ribonucleotides containing 2′-O-methyl modification to the ribose moiety.
augcuaucaccuccccugug

SEQ ID NO: 62 - RNA, 20 nt, Homo sapiens
  misc_feature (1)...(20): 2′-O-methyl ribonucleotide oligomer sequence, where A, U, G and C are modified ribonucleotides containing 2′-O-methyl modification to the ribose moiety.
ucaaguugggggacaaaaag

SEQ ID NO: 63 - RNA, 20 nt, Homo sapiens
  misc_feature (1)...(20): 2′-O-methyl ribonucleotide oligomer sequence, where A, U, G and C are modified ribonucleotides containing 2′-O-methyl modification to the ribose moiety.
agugggguggcuuuuaggau

SEQ ID NO: 64 - RNA, 20 nt, Homo sapiens
  misc_feature (1)...(20): 2′-O-methyl ribonucleotide oligomer sequence, where A, U, G and C are modified ribonucleotides containing 2′-O-methyl modification to the ribose moiety.
uuuuuaaggugugcacuuuu